(19) United States
(12) Patent Application Publication
(10) Pub. No.: US 2026/0306290 A1
(43) Pub. Date: Oct. 8, 2026

(54) Fecal Collection Systems and Methods

(71) Applicant: Sage Products LLC, Cary, IL (US)

(72) Inventors: Catherine Boulos, Cary, IL (US); David P. Beck, Cary, IL (US); Stephanie Cress, Cary, IL (US); Kaitlin T. Konopacz, Cary, IL (US); Medha Sesham, Cary, IL (US); Ronald C. Cagle, Cary, IL (US); Justin L. Vanhoozer, Cary, IL (US); Alyssa Bockman, Cary, IL (US)

(21) Appl. No.: 19/481,803

(22) PCT Filed: May 6, 2024
(86) PCT No.: PCT/US2024/028012

§ 371 Date: Nov. 5, 2025

Related U.S. Application Data
(60) Provisional application No. 63/464,460, filed on May 5, 2023.
(60) Provisional application No. 63/603,083, filed on Nov. 27, 2023.

(51) Int. Cl. A61F 5/443; A61B 10/00; A61F 5/44

(52) U.S. Cl. CPC A61F 5/443; A61F 5/4407; A61B 10/0038

(57) ABSTRACT

In an example, a fecal collection system includes a user interface, an applicator, a drainage conduit, and a collection reservoir. The applicator is coupled to a distal side of the user interface, and includes an aperture extending through a proximal side and a distal side of the applicator. The aperture of the applicator overlaps with an aperture of the user interface. The applicator further includes: a central portion extending between a superior side of the applicator and an inferior side of the applicator, a first lateral portion on a first lateral side of the central portion, a second lateral portion on a second lateral side of the central portion, and a plurality of fold lines about which the first lateral portion and the second lateral portion are configured to fold relative to the central portion.

### RELATED APPLICATIONS

The present application claims the benefit of priority to U.S. Provisional Application No. 63/603,083, filed Nov. 27, 2023, and U.S. Provisional Application No. 63/464,460, filed on May 5, 2023, the contents of which are hereby incorporated by reference in their entirety.

### FIELD

The present disclosure generally relates to fecal collection devices and systems for collecting feces discharged from the body of a user and carrying the feces away from the body.

### BACKGROUND

Under various circumstances, a user may have limited or impaired mobility such that ordinary bowel movement functions and processes are rendered difficult (or impossible). For example, a person may have impaired mobility due to a disability or may be bedridden due to an injury or illness. Also, for example, feces collection may be needed for monitoring purposes, such as for monitoring inputs and outputs in a clinical setting (e.g., in an intensive care unit, or for other clinical and/or laboratory testing).

Various approaches have been developed to address some of the problems or circumstances related to impaired or restricted bowel movement processes. However, prior approaches suffer from problems or limitations of their own.

### SUMMARY

In an example, a fecal collection system includes a user interface, an applicator, a drainage conduit, and a collection reservoir. The user interface has a proximal side and a distal side. The proximal side is configured to couple to skin of a user. The user interface includes an aperture extending through the proximal side and the distal side.

The applicator is coupled to the distal side of the user interface. The applicator includes an aperture extending through a proximal side and a distal side of the applicator. The aperture of the applicator overlaps with the aperture of the user interface. The applicator further includes: (i) a central portion extending between a superior side of the applicator and an inferior side of the applicator, where the central portion includes the aperture of the applicator, (ii) a first lateral portion on a first lateral side of the central portion, (iii) a second lateral portion on a second lateral side of the central portion, and (iv) a plurality of fold lines about which the first lateral portion and the second lateral portion are configured to fold relative to the central portion. A first subset of the plurality of fold lines are between the central portion and the first lateral portion. A second subset of the plurality of fold lines are between the central portion and the second lateral portion. Along a width extending between a first lateral side of the of the applicator and a second lateral side of the applicator, the first subset of the plurality of fold lines and the second subset of the plurality of fold lines are separated by a non-zero distance from each other. The first lateral portion and the second lateral portion are configured to move about the first subset of the plurality of fold lines and the second subset of the plurality of fold lines, respectively, to actuate the applicator between an extended state and a folded state.

The drainage conduit extends between a proximal end and a distal end. The proximal end is configured to couple to the distal side of the user interface. The drainage conduit defines a lumen configured to guide feces from the proximal end to the distal end. The collection reservoir is coupled to the distal end of the drainage conduit.

In another example, a method of using a fecal collection system includes positioning, using an applicator, a user interface at an anus of a user. The user interface has a proximal side and a distal side. The proximal side is configured to couple to skin of a user. The user interface includes an aperture extending through the proximal side and the distal side.

The applicator is coupled to the distal side of the user interface. The applicator includes an aperture extending through a proximal side and a distal side of the applicator. The aperture of the applicator overlaps with the aperture of the user interface. The applicator further includes: (i) a central portion extending between a superior side of the applicator and an inferior side of the applicator, where the central portion includes the aperture of the applicator, (ii) a first lateral portion on a first lateral side of the central portion, (iii) a second lateral portion on a second lateral side of the central portion, and (iv) a plurality of fold lines about which the first lateral portion and the second lateral portion are configured to fold relative to the central portion. A first subset of the plurality of fold lines are between the central portion and the first lateral portion. A second subset of the plurality of fold lines are between the central portion and the second lateral portion. Along a width extending between a first lateral side of the of the applicator and a second lateral side of the applicator, the first subset of the plurality of fold lines and the second subset of the plurality of fold lines are separated by a non-zero distance from each other. The first lateral portion and the second lateral portion are configured to move about the first subset of the plurality of fold lines and the second subset of the plurality of fold lines, respectively, to actuate the applicator between an extended state and a folded state.

The method also includes coupling the user interface to skin of the user by using the applicator, while the applicator in the folded state, to press the user interface against the skin of the user. Additionally, the method includes, after coupling the user interface to the skin of the user, removing the applicator from the user interface.

The features, functions, and advantages that have been discussed can be achieved independently in various embodiments or may be combined in yet other embodiments further details of which can be seen with reference to the following description and drawings.

### BRIEF DESCRIPTION OF THE FIGURES

The novel features believed characteristic of the illustrative embodiments are set forth in the appended claims. The illustrative embodiments, however, as well as a preferred mode of use, further objectives and descriptions thereof, will best be understood by reference to the following detailed description of an illustrative embodiment of the present disclosure when read in conjunction with the accompanying drawings, wherein:

FIG. 1 illustrates a simplified block diagram of a fecal collection system, according to an example.

FIG. 2 illustrates an implementation of the fecal collection system shown in FIG. 1, according to an example.

FIGS. 3A-3D illustrate a process for coupling a user interface assembly to the skin of a user according to one example implementation of the user interface assembly shown in FIG. 1.

FIG. 4 illustrates a proximal side of the user interface according to an example.

FIG. 5 illustrates a drainage conduit and a collection reservoir according to an example.

FIG. 6 illustrates an implementation of the fecal collection system shown in FIG. 1, according to another example.

FIG. 7 illustrates a distal side of an applicator for the fecal collection system shown in FIG. 6, according to an example.

FIG. 8A illustrates the user interface in a folded state for the fecal collection system shown in FIG. 6, according to an example.

FIG. 8B illustrates the user interface for the fecal collection system shown in FIG. 6, according to an example.

FIGS. 9A-9J illustrate a process for coupling the fecal collection system of FIG. 6 to a user, according to an example.

FIG. 10A illustrates a distal side of a user interface, according to an example.

FIG. 10B illustrates a proximal side of the user interface shown in FIG. 10A, according to an example.

FIG. 11A depicts a user interface, according to another example.

FIG. 11B depicts the user interface of FIG. 11A coupled to a user, according to an example.

FIG. 12A depicts a user interface assembly in a first configuration, according to another example.

FIG. 12B depicts the user interface assembly of FIG. 12A in a second configuration, according to an example.

FIG. 12C depicts the user interface assembly of FIG. 12A in the first configuration while the user interface assembly is being applied to a user, according to an example.

FIG. 13A depicts a user interface assembly in a first configuration, according to another example.

FIG. 13B depicts the user interface assembly of FIG. 13A in the first configuration while the user interface assembly is being applied to a user, according to an example.

FIG. 14 is a perspective view of an applicator, according to an example.

FIG. 15 is a first side view of the applicator shown in FIG. 14, according to the example.

FIG. 16 is a second side view of the applicator shown in FIG. 14, according to the example.

FIG. 17 is a first side view of the applicator shown in FIG. 14, according to the example.

FIG. 18 is a second side view of the applicator shown in FIG. 14, according to the example.

FIG. 19 is a first side view of the applicator shown in FIG. 14, according to the example.

FIG. 20 is a second side view of the applicator shown in FIG. 14, according to the example.

FIG. 21 depicts a user interface, according to another example.

FIG. 22A depicts a collection reservoir with a cap in a closed state according to an example.

FIG. 22B depicts the collection reservoir of FIG. 22A with the cap in an open state according to an example.

FIG. 23 depicts a flowchart for a process of using a fecal collection system according to an example.

FIG. 24A depicts a proximal side of an applicator in an extended state, according to another example.

FIG. 24B depicts a distal side of the applicator of FIG. 24A in the extended state, according to the example.

FIG. 24C shows the applicator of FIG. 24A in the folded state, according to the example.

FIG. 25A depicts a proximal side of an applicator in the extended state, according to another example.

FIG. 25B depicts a side view of the applicator of FIG. 25A in the folded state, according to the example.

FIG. 25C shows a first perspective view of the applicator of FIG. 25A in the folded state, according to the example.

FIG. 25D shows a second perspective view of the applicator of FIG. 25A in the folded state and with a user interface coupled to the proximal side of the applicator, according to the example.

FIG. 26A depicts a proximal side of an applicator in the extended state, according to another example.

FIG. 26B depicts a perspective view of the applicator of FIG. 26A in a partially folded state between the extended state and the folded state, according to the example.

FIG. 26C depicts a side view of the applicator of FIG. 26A in the folded state, according to the example.

FIG. 26D shows a perspective view of the applicator of FIG. 26A in the folded state, according to the example.

FIG. 27 depicts a flowchart for a process of using a fecal collection system according to an example.

FIG. 28 depicts a flowchart for a process of using a fecal collection system that can be used with the process of FIG. 27 according to an example.

FIG. 29 depicts a flowchart for a process of using a fecal collection system that can be used with the process of FIG. 27 according to an example.

FIG. 30 depicts a flowchart for a process of using a fecal collection system that can be used with the process of FIG. 27 according to an example.

FIG. 31A depicts a collection reservoir, according to an example.

FIG. 31B depicts a second one-way valve for the collection reservoir shown in FIG. 31A, according to the example.

FIG. 32A depicts a second valve, according to an example.

FIG. 32A depicts an exploded view of the user interface, according to an example.

FIG. 32B depicts a view of a proximal side of the user interface shown in FIG. 32A, according to the example.

FIG. 33A depicts a side view of an applicator and a user interface, according to an example.

FIG. 33B depicts a top view of the applicator and the user interface of FIG. 33A, according to the example.

FIG. 34A depicts a perspective view of an applicator and a user interface, according to another example.

FIG. 34B depicts the applicator of FIG. 34A pressing the user interface against a user, according to an example.

FIG. 35A depicts a first perspective view of an applicator and a user interface, according to another example.

FIG. 35B depicts a second perspective view of an applicator and a user interface, according to another example.

FIG. 36 depicts a perspective view of an applicator and a user interface, according to another example.

FIG. 37 depicts a perspective view of an applicator and a user interface, according to another example.

FIG. 38A depicts a first perspective view of an applicator and a user interface, according to another example.

FIG. 38B depicts a second perspective view of an applicator and a user interface, according to another example.

FIG. 39A depicts a perspective view of an applicator, according to another example.

FIG. 39B depicts a perspective view of an applicator, according to another example.

FIG. 39C depicts a perspective view of an applicator, according to another example.

FIG. 39D depicts a perspective view of an applicator, according to another example.

FIG. 39E depicts a perspective view of an applicator, according to another example.

FIG. 39F depicts a side view of an applicator, according to another example.

FIG. 40 depicts a perspective view of an applicator, according to another example.

FIG. 41A depicts a perspective view of an applicator, according to another example.

FIG. 41B depicts a lateral side view of the applicator shown in FIG. 41A, according to the example.

FIG. 41C depicts a proximal side view of the applicator shown in FIG. 41A, according to the example.

FIG. 42A depicts an applicator in a laid flat configuration, according to an example.

FIG. 42B depicts the applicator of FIG. 42B in a folded configuration, according to an example.

FIG. 43A depicts a perspective view of an applicator, according to another example.

FIG. 43B depicts a perspective view of an applicator, according to another example.

FIG. 43C depicts a perspective view of an applicator, according to another example.

FIG. 43D depicts a perspective view of an applicator, according to another example.

FIG. 43E depicts a perspective view of an applicator, according to another example.

### DETAILED DESCRIPTION

Disclosed embodiments will now be described more fully hereinafter with reference to the accompanying drawings, in which some, but not all of the disclosed embodiments are shown. Indeed, several different embodiments may be described and should not be construed as limited to the embodiments set forth herein. Rather, these embodiments are described so that this disclosure will be thorough and complete and will fully convey the scope of the disclosure to those skilled in the art.

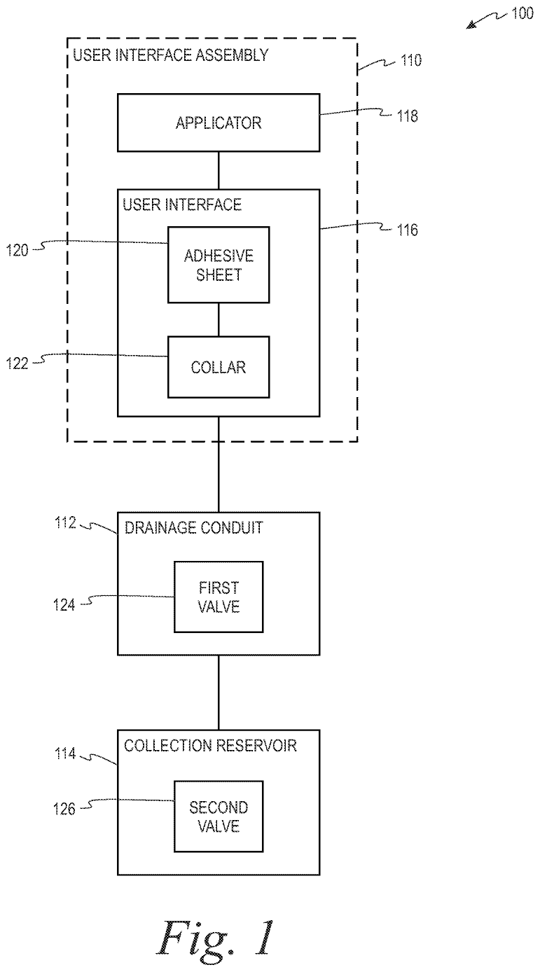

Referring to FIG. 1, a simplified block diagram of a fecal collection system 100 is shown according to an example. As shown in FIG. 1, the fecal collection system 100 includes a user interface assembly 110, a drainage conduit 112, and a collection reservoir 114. The fecal collection system 100 can be coupled to the skin of the user such that the fecal collection system 100 is external to the body of the user.

The user interface assembly 110 includes a user interface 116 and an applicator 118. The user interface 116 has a proximal side and a distal side. The proximal side is configured to couple to the skin of a user. For instance, the user interface 116 can include an adhesive sheet 120 on the proximal side. As one example, the adhesive sheet 120 can include a biocompatible adhesive such as, for instance, a silicon adhesive that can provide for coupling the user interface 116 to the skin of the user. In one example, the adhesive sheet 120 can be configured to couple the user interface 116 to the user's skin for a period of at least eight hours before being removed and/or replaced. In another example, the adhesive sheet 120 can be configured to couple the user interface 116 to the user's skin for a period of approximately 12 to 72 hours before being removed and/or replaced.

The applicator 118 is coupled to the distal side of the user interface 116. The applicator 118 is removable from the user interface 116 while the user interface 116 is coupled to the user. In this arrangement, the applicator 118 can assist in coupling the user interface 116 to the skin of the user, and then be removed to provide for coupling the drainage conduit 112 to the user interface 116 and/or reduce an amount of material of the fecal collection system 100 between the user and a support structure (e.g., a hospital bed and/or wheel chair). Removing the applicator 118 can thus help to mitigate a risk of pressure sores.

As examples, the applicator 118 can be formed from at least one material selected from a group consisting of: a foam, cardstock, cardboard, and chipboard (e.g., a material made from wood pulp and omitting corrugated channels of cardboard). In examples in which the applicator 118 is formed from a foam, the foam material can provide for improved conformation of the applicator 118 and the user interface 116 to the skin of the user. Cardstock can be biodegradable, which may be a benefit in examples in which the applicator 118 is formed from cardstock.

The drainage conduit 112 extends between a proximal end and a distal end. In one example, the proximal end of the drainage conduit 112 can be fixedly coupled to the distal side of the user interface 116 before the applicator 118 is removed from the user interface 116. In this example, the drainage conduit 112 can be coupled to the user interface 116 while the user interface 116 is being coupled to the skin of the user. In another example, the proximal end is configured to couple to the distal side of the user interface 116 after the applicator 118 is removed from the user interface 116. More generally, the drainage conduit 112 defines a lumen configured to guide feces from the proximal end to the distal end. The collection reservoir 114 coupled to the distal end of the drainage conduit 112. The collection reservoir 114 is configured to contain the feces. For instance, the collection reservoir 114 can be made from a fluid impermeable material. As examples, the collection reservoir 114 can be made from silicone, rubber, latex, and/or a thermoplastic.

The drainage conduit 112 and the lumen can have an elongated shape. In an example, the drainage conduit 112 can have a length of approximately 4 feet to approximately 6 feet. This length can help to position the collection reservoir 114 at a location that is remote from the user (e.g., at a side of a patient support structure such as a hospital bed). However, the drainage conduit 112 can have a different length in other examples.

Additionally, in some examples, the drainage conduit 112 can be configured to guide the feces from the proximal end to the distal end responsive to milking the drainage conduit 112 toward the collection reservoir 114 (e.g., by applying compression to the drainage conduit 112 along a length of drainage conduit 112 in a direction from the proximal end to the distal end). For instance, the drainage conduit 112 can have a hardness and/or a pliability that facilitates manually milking the feces toward the collection reservoir 114. Also, as examples, the drainage conduit 112 can be formed from a polyurethane (PU) and/or a polyvinyl chloride (PVC). For instance, the drainage conduit 112 can be a film tube having a thickness between approximately 2 mil and approximately 4 mil.

In some implementations, the drainage conduit 112 can be at least partially transparent or translucent. This can help to determine whether feces is present in the drainage conduit 112 such that a determination can be made to milk the drainage conduit 112 and force the feces to the collection reservoir 114.

In an alternative example, the fecal collection system 100 can omit the drainage conduit 112 such that the collection reservoir 114 is directly coupled to the user interface 116. Although this may reduce a cost of manufacture and reduce a need to milk the drainage conduit 112, the collection reservoir 114 is positioned nearer to the user, which increases a bulk of the device that may contact the user and makes replacing the collection reservoir 114 more challenging.

As noted above, the user interface 116 can include the adhesive sheet 120. The adhesive sheet 120 can define an aperture in communication with the lumen of drainage conduit 112. In this arrangement, feces can pass from an anus of the user into the drainage conduit 112 through the aperture of the adhesive sheet 120. The drainage conduit 112 can then guide the feces to the collection reservoir 114.

In one example, the aperture of the adhesive sheet 120 can have a diameter of approximately 1.5 inches. This can help to provide a sufficiently large opening to receive the feces into the drainage conduit 112 while allowing for a surface area of the adhesive sheet 120 to secure the fecal collection system 100 to the skin of the user. In another example, the aperture of the adhesive sheet 120 can have a diameter of approximately 1 inch to approximately 2 inches.

In some implementations, the user interface 116 can also include a collar 122 extending distally from the adhesive sheet 120. As described above, the adhesive sheet 120 can include an adhesive such as, for example, a silicon adhesive that can provide for coupling the user interface 116 to the skin of the user. The proximal end of the drainage conduit 112 can be coupled to the collar 122 of the user interface 116. For example, the collar 122 can be a foam wedge and can be received at the proximal end of the drainage conduit 112. In this example, the collar 122 can thus couple to the drainage conduit 112 by friction-fit connection. However, in other examples, the collar 122 can additionally or alternatively couple to the drainage conduit 112 by a threaded connection, a hook and loop connection, and/or an adhesive connection.

More generally, the collar 122 can be configured to provide for coupling, decoupling, and/or recoupling one or more drainage conduits 112 to the user interface 116. As such, the collar 122 can provide for changing the drainage conduit 112 and/or the collection reservoir 114 without removing the user interface from 116 from the user. For instance, a method of using the fecal collection system 100 can include removing a first drainage conduit 112 from the user interface 116 after an initial period of use (e.g., approximately 8 hours) and then coupling a second drainage conduit 112 to the user interface 116.

In an example, the collar 122 can define a first aperture in communication with the lumen of drainage conduit 112, and the adhesive sheet 120 can define a second aperture that is axially aligned with the first aperture of the collar 122. In this arrangement, feces can pass from an anus of the user into the drainage conduit 112 through the first aperture of the collar 122 and the second aperture of the adhesive sheet 120. The drainage conduit 112 can then guide the feces to the collection reservoir 114.

In one example, the first aperture and the second aperture can have a diameter of approximately 1.5 inches. This can help to provide a sufficiently large opening to receive the feces into the drainage conduit 112 while allowing for a surface area of the adhesive sheet 120 to secure the fecal collection system 100 to the skin of the user. In another example, the first aperture and/or the second aperture can have a diameter of approximately 1 inches to approximately 2 inches.

In an example, the fecal collection system 100 can also include a first one-way valve 124 in the lumen defined by the drainage conduit 112. The first one-way valve 124 is configured to allow feces to flow in a direction from the proximal end to the distal end of the drainage conduit 112 and inhibit the feces from flowing in a direction from the distal end to the proximal end of the drainage conduit 112. This can help inhibit feces from migrating back toward and into contact with the skin of the user.

In one example, the first one-way valve 124 can be configured to transition from a closed state to an open state responsive to feces passing through the first one-way valve 124 in a distal direction (e.g., from the user toward the drainage conduit 112). In the closed state, the first one-way valve 124 can inhibit backflow of feces in a proximal direction toward the user (e.g., toward the user interface assembly 110). In the open state, the first one-way valve 124 can expand to define an enlarged passageway that allows the feces to pass through the first one-way valve 124 in a distal direction. In one implementation, the first one-way valve 124 can be configured to expand to have a diameter that is approximately equal to a diameter of the drainage conduit 112. For instance, in an implementation in which the drainage conduit 112 has a diameter of approximately 1.5 inches, the first one-way valve 124 can expand from a diameter of approximately zero inches in the closed state to a diameter of approximately 1.5 inches in the open state.

In one example implementation, the first one-way valve 124 can include a plurality of flaps that extend distally from a proximal end of the drainage conduit 112 (e.g., at the aperture of the adhesive sheet 120). In this arrangement, the flaps can taper towards each other along a length of the flaps in a direction from the proximal end of the drainage conduit 112 toward a distal end of the drainage conduit 112. In one example, a material of the flaps can be the same as a material of the drainage conduit 112. The flaps can a monolithic structure (e.g., integral) with the drainage conduit 112 or the flaps can be distinct structures coupled to the drainage conduit 112 (e.g., via a weld).

The collection reservoir 114 can additionally or alternatively include a second one-way valve 126 that is configured to allow feces to flow from the drainage conduit 112 to the collection reservoir 114 and inhibit the feces from flowing from the collection reservoir 114 to the drainage conduit 112. This can also help inhibit feces from migrating back toward and into contact with the skin of the user. In one example, the drainage conduit 112 can have a diameter of approximately 1 inch to approximately 3 inches at the distal end 212B.

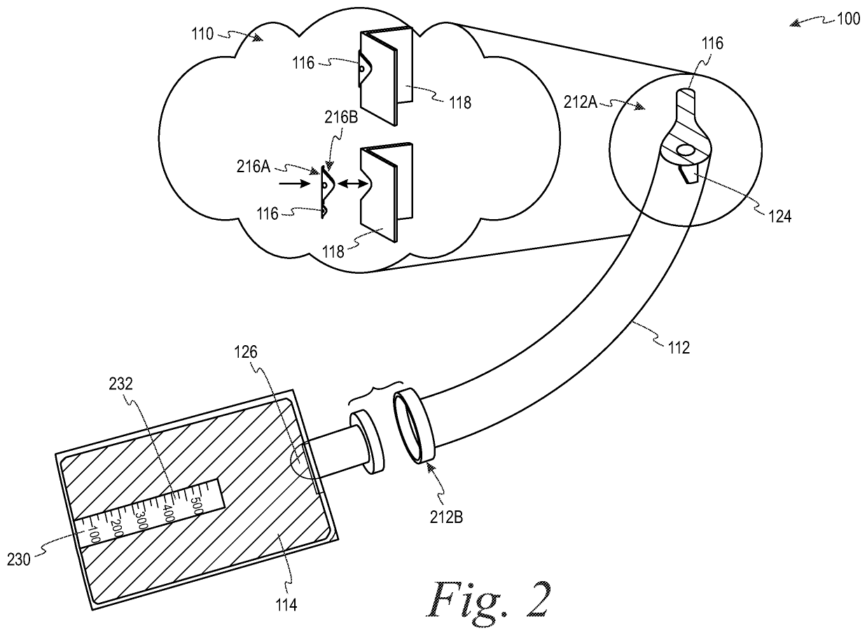

FIG. 2 depicts an implementation of the fecal collection system 100 according to an example. As shown in FIG. 2, the fecal collection system 100 includes the user interface assembly 110, the drainage conduit 112, and the collection reservoir 114 as described above. As described above, the user interface assembly 110 includes the user interface 116 having a proximal side 216A and a distal side 216B. Additionally, in FIG. 2, the drainage conduit 112 extends between a proximal end 212A and a distal end 212B.

As shown in FIG. 2, the drainage conduit 112 can be configured to be coupled, decoupled, and re-coupled to the collection reservoir 114. This can provide for decoupling a first collection reservoir 114 containing feces, and coupling a second collection reservoir 114 that is empty to continue collecting feces without having to decouple the user interface 116 and/or the drainage conduit 112 from the skin of the user. In some examples, the drainage conduit 112 and the collection reservoir 114 can be coupled, decoupled, and recoupled to the user interface 116 while the user interface 116 remains coupled to the skin of the user. This can provide for changing the drainage conduit 112 and/or the collection reservoir 114 without removing the user interface from 116 from the user. In other examples, the drainage conduit 112 can be permanently fixed to the collection reservoir 114. This can help to mitigate a risk of leakage at an interface between the user interface 116 and the drainage conduit 112.

Additionally, as shown in FIG. 2, the collection reservoir 114 can include a measurement window 230 including a plurality of volumetric markings 232. The measurement window 230 can be at least one of transparent or translucent such that a quantity of feces in the collection reservoir 114 can be visually observed. This can provide an indication when the collection reservoir 114 should be changed, and/or when sufficient volume of feces has been collected to provide a sample for a diagnostic evaluation.

In FIG. 2, the first one-way valve 124 is positioned at the proximal end 214A. In other examples, the first one-way valve 124 can be positioned between the proximal end 214A and the distal end 214B of the drainage conduit 112.

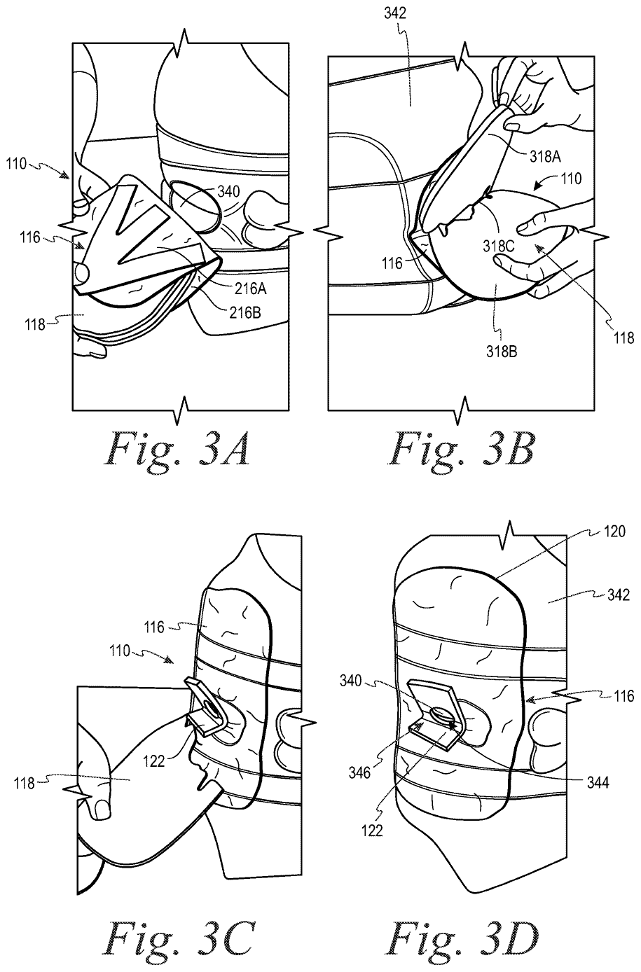

FIGS. 3A-3D depict a process for coupling the user interface assembly 110 to the skin of a user according to one example implementation of the user interface assembly 110. As shown in FIGS. 3A-3D, the user interface assembly 110 includes the user interface 116 and the applicator 118. In FIG. 3A, the user interface assembly 110 is positioned at an anus 340 of the user. The user interface assembly 110 is folded such that the proximal side 216A of the user interface 116 faces outward, and the distal side 216B of the user interface 116 faces inward.

In FIG. 3B, the applicator 118 is used to press the proximal side 216A of the user interface 116 against the skin 342 of the user. In this example, the applicator 118 includes a first lateral portion 318A and a second lateral portion 318B that are hingedly movable relative to each other. For instance, the applicator 118 can include a hinged portion 318D between the first lateral portion 318A and the second lateral portion 318B. In this example, the hinged portion 318C is perforated to assist in removing the applicator 118 from the user interface 116. As shown in FIG. 3B, the first lateral portion 318A of the applicator 118 and a second lateral portion 318B of the applicator 118 can be moved relative to each other to press the user interface 116 against the skin 342 of the user.

In FIG. 3C, the applicator 118 is being removed from the user interface 116. For example, the applicator 118 can be removed from the user interface 116 by decoupling the first lateral portion 318A and the second lateral portion 318B from the user interface 116 along the perforation line of the hinged portion 318C. As shown in FIGS. 3C-3D, the removing the applicator 118 can expose the collar 122 of the user interface 116. In FIG. 3D, the user interface 116 is coupled to the skin 342 of the user. As shown in FIG. 3D, the adhesive sheet 120 is coupled to the skin 342 and the collar 122 extends distally from the adhesive sheet 120. Also, in FIG. 3D, the collar 122 defines a first aperture 344, the adhesive sheet 120 defines a second aperture 346, and the first aperture 344 and the second aperture 346 are aligned with the anus 340 of the user.

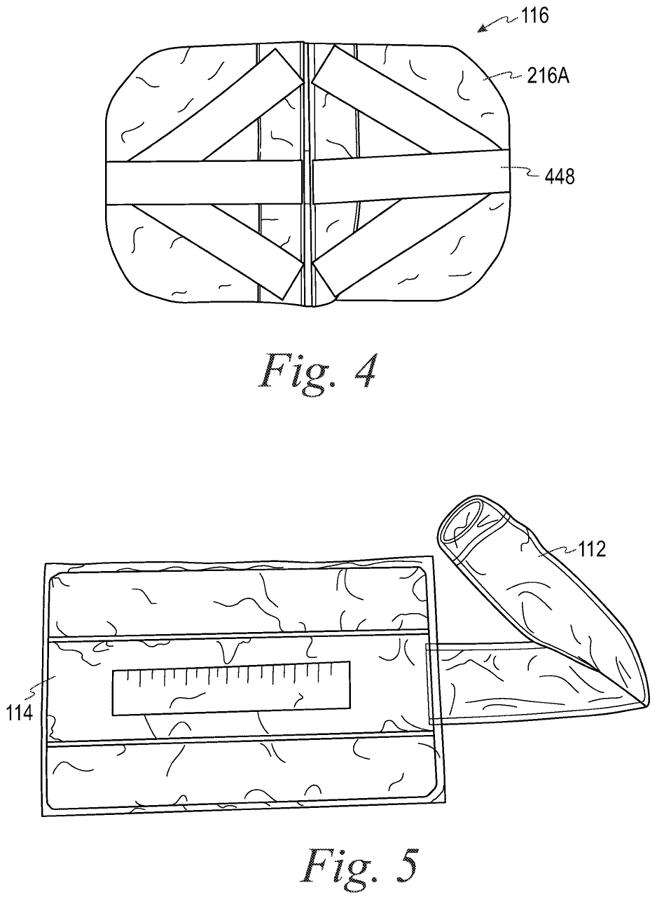

FIG. 4 depicts the proximal side 216A of the user interface 116 according to an example. In FIG. 4, the user interface 116 includes a release liner 448 that covers and adhesive on the proximal side 216A of the user interface 116. The release liner 448 can be removed to expose the adhesive prior to coupling the user interface 116 to the skin of the user.

FIG. 5 depicts the drainage conduit 112 and the collection reservoir 114 according to an example implementation.

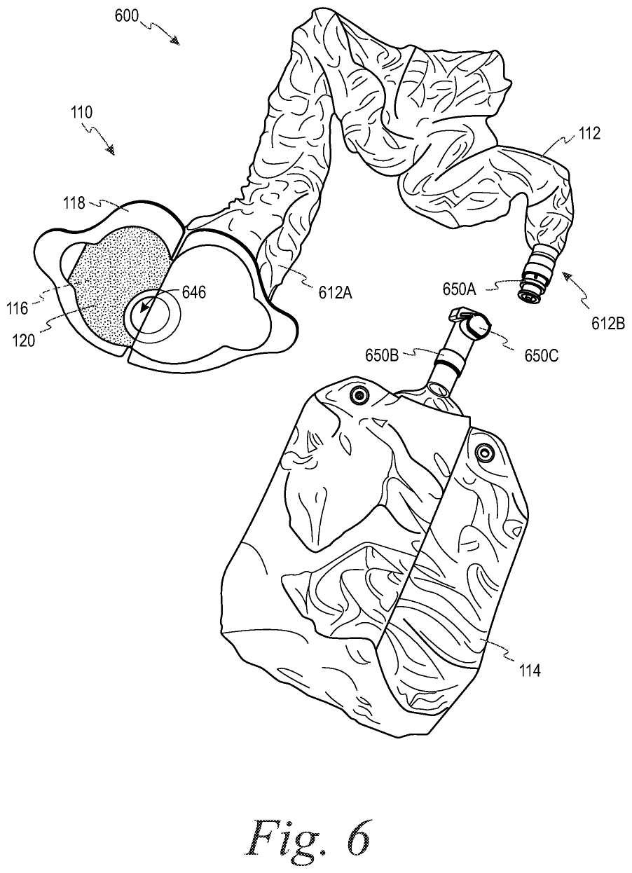

Referring now to FIG. 6, a fecal collection system 600 is depicted for an implementation of the fecal collection system 100 according to another example. As shown in FIG. 6, the fecal collection system 600 includes the user interface assembly 110, the drainage conduit 112, and the collection reservoir 114 as described above. As described above, the user interface assembly 110 includes the user interface 116 having a proximal side and a distal side. Additionally, in FIG. 6, the drainage conduit 112 extends between a proximal end 612A and a distal end 612B.

As shown in FIG. 6, the drainage conduit 112 can be configured to be coupled, decoupled, and re-coupled to the collection reservoir 114. This can provide for decoupling a first collection reservoir 114 containing feces, and coupling a second collection reservoir 114 that is empty to continue collecting feces without having to decouple the user interface 116 from the skin of the user. In other examples, the drainage conduit 112 can be permanently fixed to the collection reservoir 114. In such examples, the drainage conduit 112 and the collection reservoir 114 can be coupled, decoupled, and recoupled to the user interface 116 while the user interface 116 remains coupled to the skin of the user (e.g., in other examples, the user interface 116 can further include the collar 122 described above).

In FIG. 6, the distal end 612B of the drainage conduit 112 includes a male quick-connect coupler 650A that can couple to a female quick-connect coupler 650B of the collection reservoir 114. The female quick-connect coupler 650B of the collection reservoir 114 further includes an actuator 650C (e.g., a button) that is operable to couple and/or decouple the male quick-connect coupler 650A and the female quick-connect coupler 650B to each other. In this arrangement, the drainage conduit 112 can remain securely coupled to the collection reservoir 114 until the actuator 650C is operated to decouple the male quick-connect coupler 650A of the drainage conduit 112 from the female quick-connect coupler 650B of the collection reservoir 114. This can help to inhibit (or prevent) inadvertent decoupling of the drainage conduit 112 and the collection reservoir 114. Although the drainage conduit 112 includes the male quick-connect coupler 650A and the collection reservoir 114 includes the female quick-connect coupler 650B in FIG. 6, the drainage conduit 112 can include the female quick-connect coupler 650B and the collection reservoir 114 can include the male quick-connect coupler 650A in an alternative example.

Additionally, as described above, the collection reservoir 114 can include a measurement window including a plurality of volumetric markings (e.g., similar to the collection reservoir 114 shown in FIGS. 2 and 5). The measurement window can be at least one of transparent or translucent such that a quantity of feces in the collection reservoir can be visually observed. This can provide an indication when the collection reservoir should be changed, and/or when sufficient volume of feces has been collected to provide a sample for a diagnostic evaluation.

In FIG. 6, the proximal end 612A of the drainage conduit 112 is coupled to the distal side of the user interface 116. In this example, the drainage conduit 112 can be non-removably coupled (e.g., fixedly and permanently coupled) to the user interface 116. For instance, the proximal end 612A of the drainage conduit 112 can be welded to the adhesive sheet 120 at an aperture 646 in the adhesive sheet 120. Additionally, the applicator 118 can include an aperture that overlaps with the aperture 646 in the user interface 116 such that the drainage conduit 112 can extend through an aperture in the applicator 118 to couple to the user interface 116 with the lumen of the drainage conduit 112 aligned with the aperture in the user interface 116.

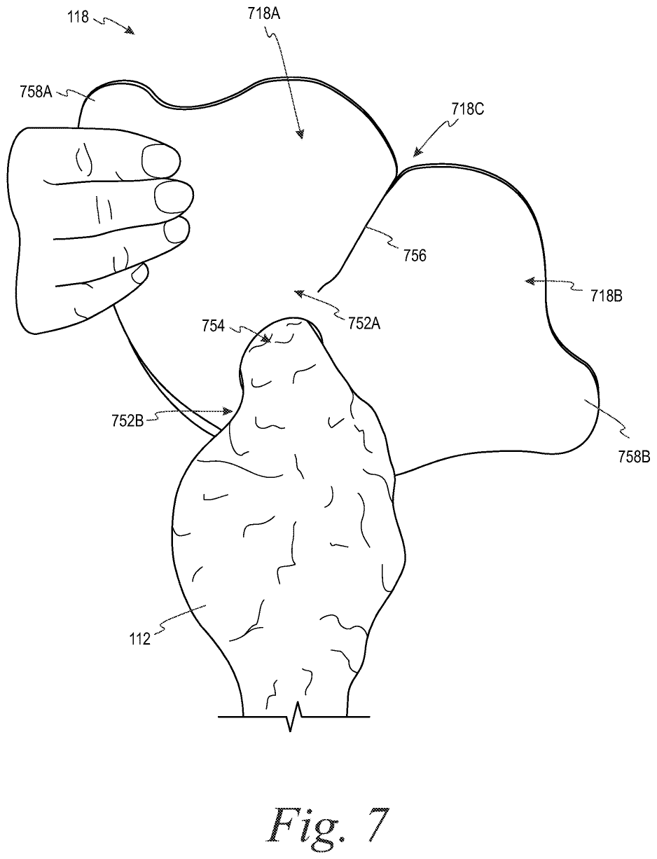

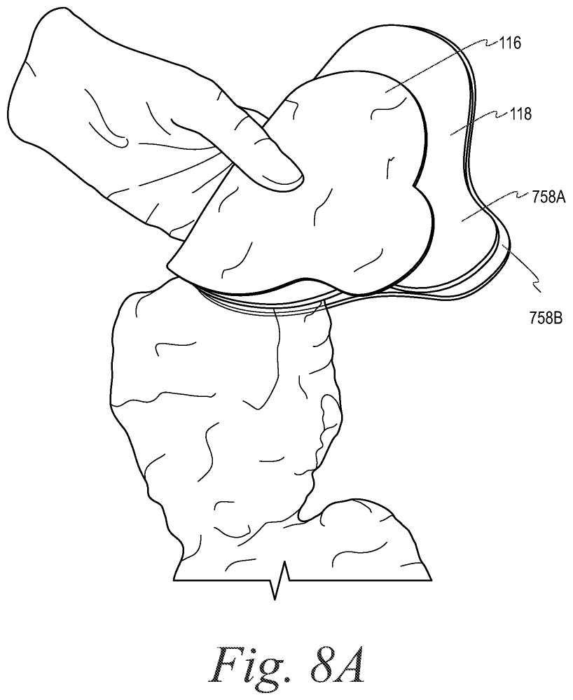

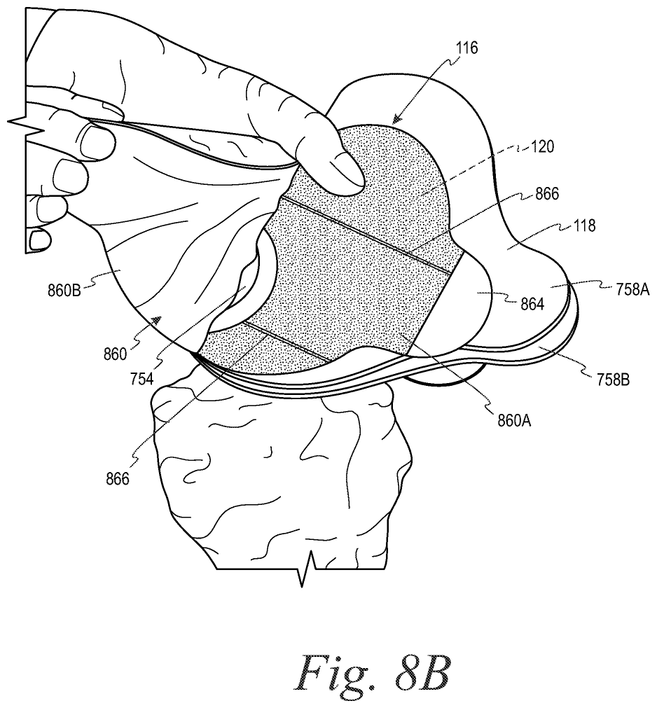

FIG. 7 depicts the applicator 118 according to the example shown in FIG. 6. As shown in FIG. 7, the applicator 118 includes a first lateral portion 718A, a second lateral portion 718B, and a hinge portion 718C between the first lateral portion 718A and the second lateral portion 718B. In this arrangement, the first lateral portion 718A and the second lateral portion 718B are configured to move about a linear axis defined by the hinge portion 718C between an extended state (as shown in FIG. 7) and a folded state (as shown in FIGS. 8A-8B). As described below, the folded state can help to insert the applicator 118 and the user interface 116 between buttocks to access an anus of a user. The extended state can help remove the applicator 118 from the user interface 116, as described above.

The hinge portion 718C is further configured to facilitate removing the applicator 118 from the user interface 116. For example, in FIG. 7, the hinge portion 718C includes a first bridge portion 752A and a second bridge portion 752B connecting the first lateral portion 718A of the applicator 118 to the second lateral portion 718B of the applicator 118 (e.g., the first later portion 718A and the second lateral portion 718B can be coupled to each other only at the first bridge portion 752A and the second bridge portion 752B). The first bridge portion 752A and the second bridge portion 752B can have a size that is relatively small compared to an overall size of the applicator 718 in a dimension extending along an intersection between the first lateral portion 718A and the second lateral portion 718B. For instance, a combined length of the first bridge portion 752A and the second bridge portion 752B can be less than approximately 50 percent of an overall length of the applicator 118 in this dimension. This relative size can provide for (i) inhibiting inadvertent separation of the first lateral portion 718A and the second lateral portion 718B during coupling of the user interface 116 to the user and (ii) easily tearing the first lateral portion 718A and the second lateral portion 718B apart from each other after coupling the user interface 116 to the user.

In some implementations, the first bridge portion 752A and/or the second bridge portion 752B can include perforations to assist in decoupling the first lateral portion 718A and the second lateral portion 718B from each other. The perforations can allow for the combined length of the first bridge portion 752A and the second bridge portion 752B to be greater than approximately 50 percent of an overall length of the applicator 118 in the dimension extending along an intersection between the first lateral portion 718A and the second lateral portion 718B (e.g., along the hinge portion 718C).

Additionally, as shown in FIG. 7, the drainage conduit 112 extends through an aperture 754 in the applicator 118. The first bridge portion 752A is defined between the aperture 754 and a slit 756, which extends along the hinge portion 718C between the first lateral portion 718A and the second lateral portion 718B of the applicator 718. The slit 756 can help to reduce a size of the first bridge portion 752A relative to the overall size of the applicator 718 along the interface between the first lateral portion 718A and the second lateral portion 718B. The slit 756 can additionally or alternatively help to enhance the movability of the first lateral portion 718A and the second lateral portion 718B about the hinge portion 752C.

In one example, the slit 756 can have a length that is greater than approximately 40 percent of the overall length of the applicator 118 in the dimension extending along the hinge portion 718C (e.g., a dimension through the first bridge portion 752A, the aperture 754, and the second bridge portion 752B). This can help to achieve the functionalities of the slit 756 described above.

The second bridge portion 752B can be defined between the aperture 754 (e.g., at a point that is opposite the first bridge portion 752A) and a peripheral edge of the applicator 118 at the hinge portion 718C. As shown in FIG. 7, the aperture 754 can be at a location that is offset from a center of the applicator 118. More particularly, the aperture 754 can be at position that is closer to a lower edge of the applicator 118 than an upper edge of the applicator 118. As such, the proximal end of the drainage conduit 112 can be coupled to the user interface 116 at a location that is offset below a center of the user interface 116. This can help to apply the user interface 116 at the anus of the user, which is generally located at a position that is below a center of the user's buttocks.

As shown in FIG. 7, the applicator 118 can further include a first tab 758A on a peripheral edge of the first lateral portion 718A and a second tab 758B on a peripheral edge of the second lateral portion 718B. The first tab 758A and the second tab 758B can extend laterally past a peripheral edge of the user interface 116 such that the first tab 758A and the second tab 758B can facilitate gripping and handling of the applicator 118 when positioning the user interface assembly 110 between a user's buttocks.

FIGS. 8A-8B depict the user interface 116 and the applicator 118 in the folded state such that the first lateral portion 718A and the second lateral portion 718B are folded about the hinge portion 718C, and the user interface 116 is positioned externally to the applicator 118. As shown in FIG. 8A, the applicator 118 can have a size that is larger than the user interface 116 (e.g., the first tab 758A and the second tab 758B can extend past a peripheral edge of the user interface 116). This can help provide access to the applicator 118. In another example, the applicator can have a size that is approximately equal to a size of the user interface 116. More generally, the user interface 116 and the applicator 118 can be configured to provide for holding the user interface 116 and the applicator 118 in a single hand while the user interface 116 and the applicator 118 are in a folded state.

In FIGS. 8A-8B, the user interface 116 includes the adhesive sheet 120 (shown in FIG. 8B), a first release liner 860, and a second release liner (not shown in FIGS. 8A-8B). The first release liner 860 is removably coupled to a first half of the adhesive sheet 120 (e.g., on a first side of the hinge portion 718C and the aperture 754 in FIGS. 8A-8B), and the second release liner is removably coupled to a second half of the adhesive sheet 120 (e.g., on a second side of the hinge portion 718C and the aperture 754 in FIGS. 8A-8B). In this arrangement, the first release liner 860 and the second release liner can be independently removed from the adhesive sheet 120 to provide for coupling the user interface 116 to the user's skin on one side at a time.

The first release liner 860 can include a first portion 860A and a second portion 860B. The first portion 860A can cover the first half of the adhesive sheet 120 to inhibit the adhesive sheet 120 prematurely or inadvertently adhering to an object prior to application of the user interface 116 to the user. The second portion 860B can have a first end that is coupled to the first portion 860A at an inner portion of the user interface 116 (e.g., at the aperture 754), and a second end that is separate from the first portion 860A at an outer portion of the user interface 116 (e.g., at the peripheral edge of the user interface 116). In this arrangement, a practitioner can grasp the second end at the periphery of the user interface 116 and pull outwardly away from the user to separate the first portion 860A of the first release liner 860 from the adhesive sheet 120. In this way, the first release liner 860 can be progressively expose the first half the adhesive sheet 120 in a direction from the aperture 754 towards the peripheral edge of the user interface 116. This can help to improve coupling the adhesive sheet 120 to the buttocks of the user with the aperture 754 aligned with the anus of the user.

Although not shown in FIGS. 8A-8B, the second release liner can be arranged in a similar manner with respect to the second half of the adhesive sheet 120. As such, the second release liner can include a first portion and a second portion. The first portion of the second release liner can cover the second half of the adhesive sheet 120 to inhibit the adhesive sheet 120 prematurely or inadvertently adhering to an object prior to application of the user interface 116 to the user. The second portion can have a first end that is coupled to the first portion at an inner portion of the user interface 116 (e.g., at the aperture 754), and a second end that is separate from the first portion at an outer portion of the user interface 116 (e.g., at the peripheral edge of the user interface 116). In this arrangement, a practitioner can grasp the second end at the periphery of the user interface 116 and pull outwardly away from the user to separate the first portion of the second release liner from the adhesive sheet 120. In this way, the second release liner can be progressively expose the second half the adhesive sheet 120 in a direction from the aperture 754 towards the peripheral edge of the user interface 116. This can help to improve the coupling the adhesive sheet 120 to the buttocks of the user with the aperture 754 aligned with the anus of the user.

As shown in FIG. 8B, the first half and the second half of the adhesive sheet 120 can each include a grip region 864. The grip region 864 can be located at the outer periphery of the adhesive sheet 120 and omit the adhesive. This can allow a practitioner to hold the grip region 864 to manipulate and handle the adhesive sheet 120 while applying the adhesive sheet 120 to the user's buttocks. The grip region 864 can also facilitate easier removal of the adhesive sheet 120 from the user as compared to an alternative implementation in which the adhesive extends over an entire surface area of the adhesive sheet 120.

Additionally, as shown in FIG. 8B, the user interface 116 and/or the applicator 118 can include an alignment indicator 866 that can help to align the aperture 754 and the drainage conduit 112 with the anus of the user. For example, in FIG. 8B, the alignment indicator 866 includes a first line that extends on the applicator 118 from a top of the aperture 754 to the peripheral edge of the adhesive sheet 120, and a second line that extends on the applicator 118 from a bottom of the aperture 754 to the peripheral edge of the adhesive sheet 120. In this arrangement, the alignment indicator 866 can provide a visual indication of the location of the aperture 754 of the applicator 118, the aperture 646 of the user interface 116 (shown in FIG. 6), and the drainage conduit 112 positioned between the buttocks even though the aperture 754 and the aperture 646 cannot be directly visualized.

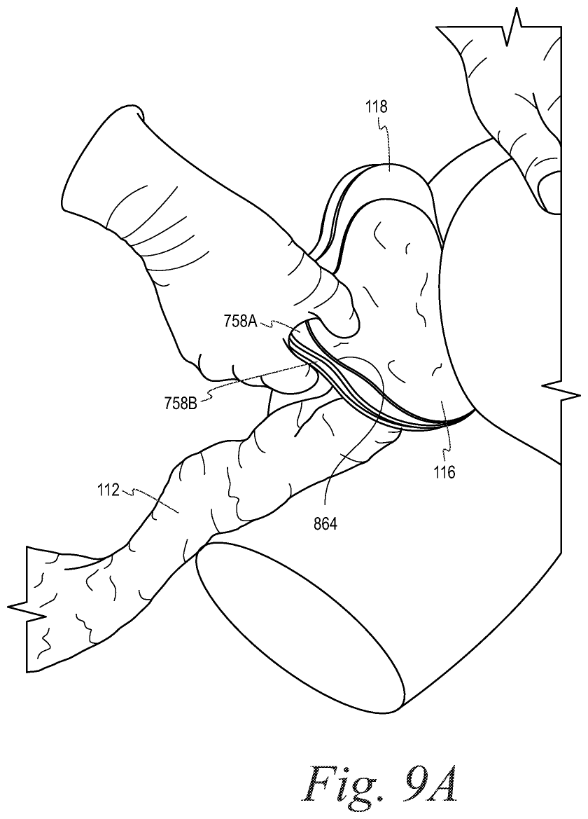

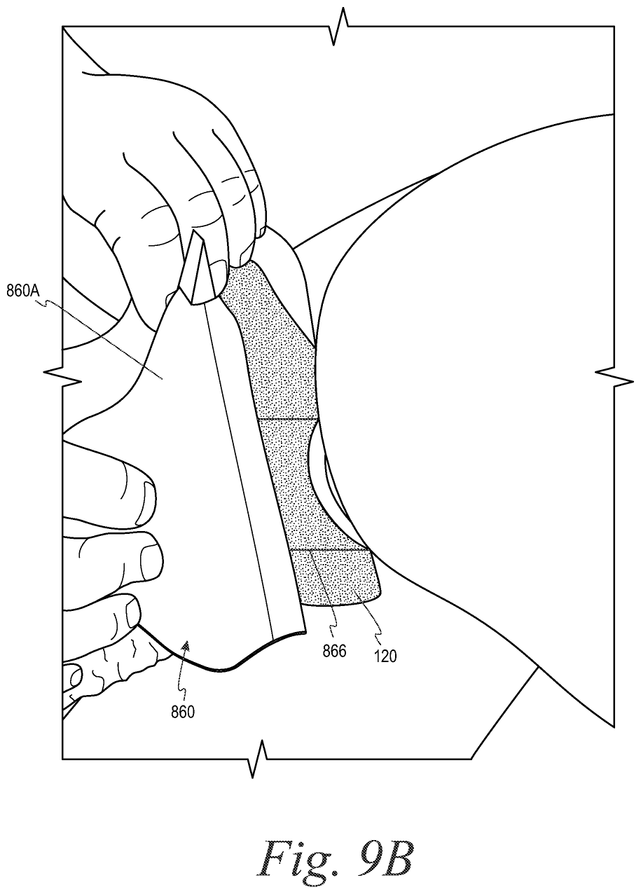

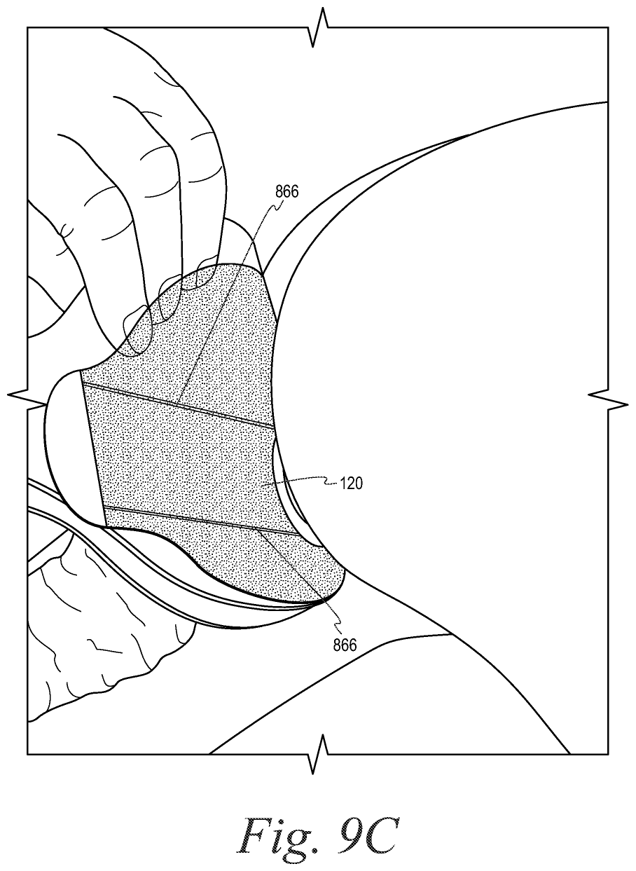

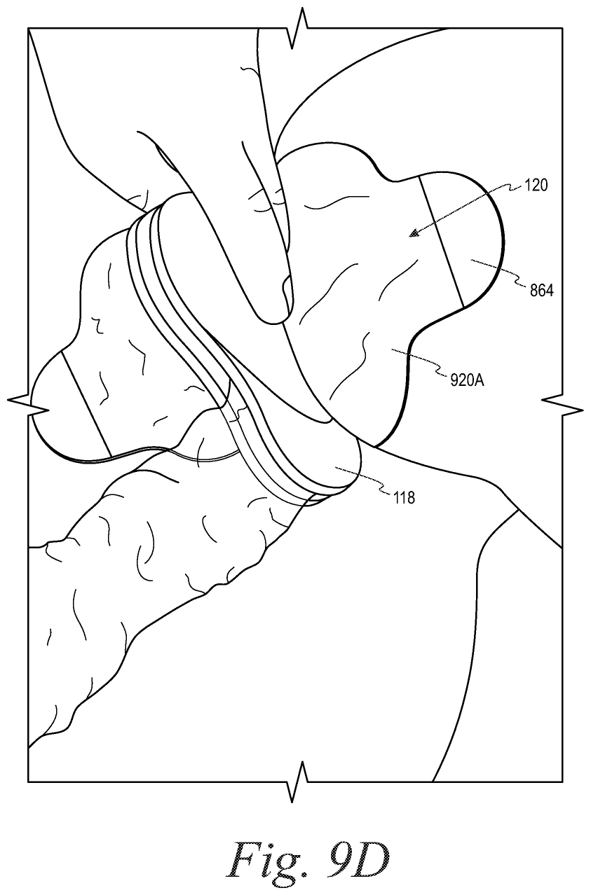

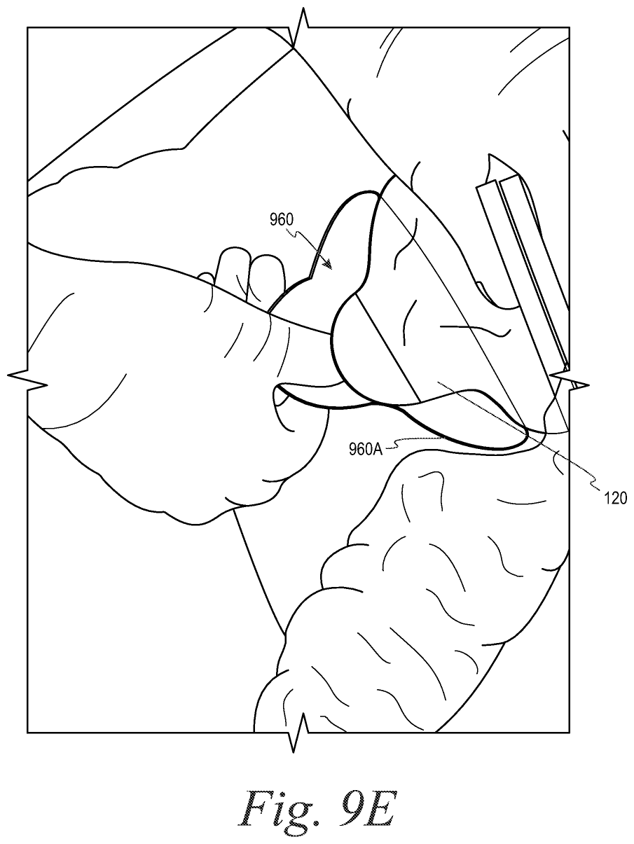

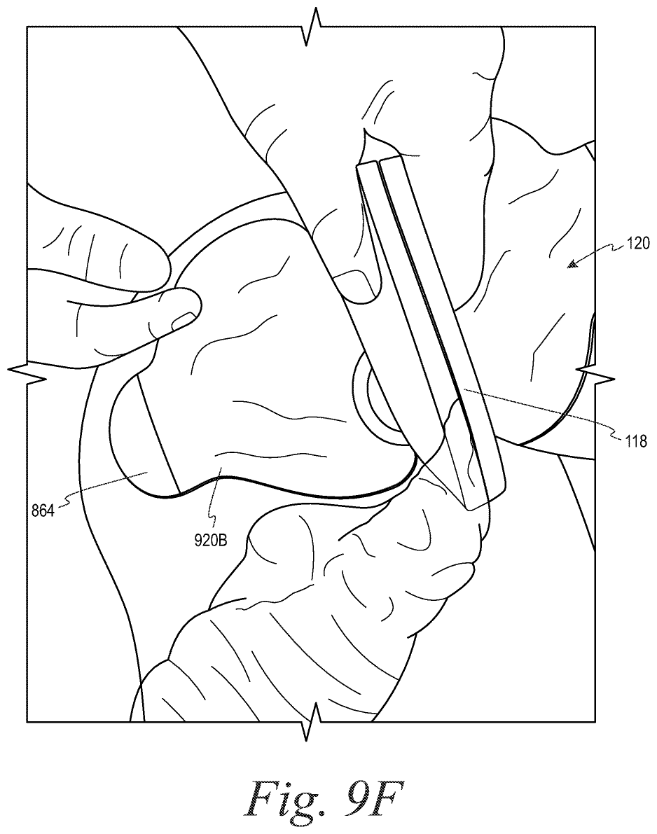

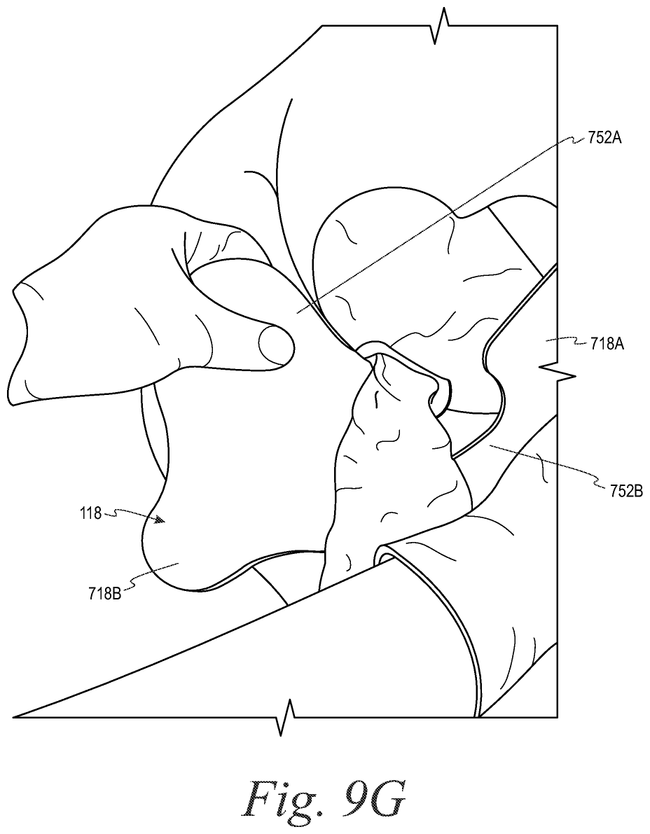

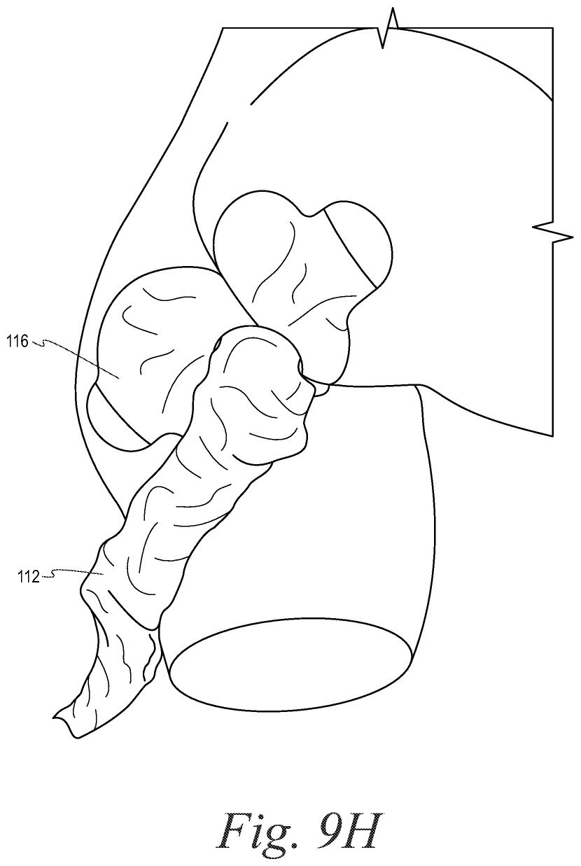

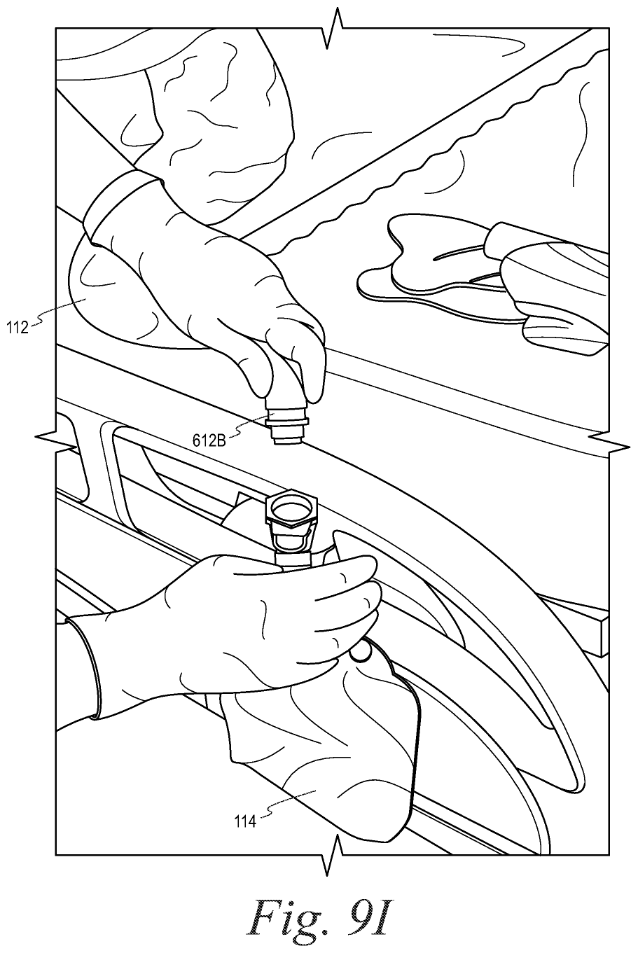

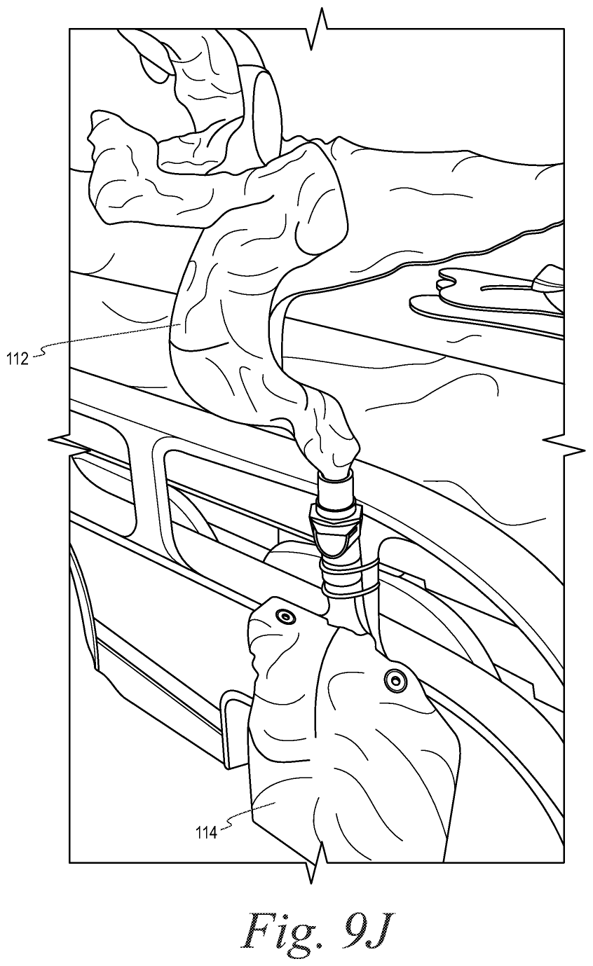

Referring now to FIGS. 9A-9J, process for coupling the fecal collection system 600 to a user is depicted according to one example implementation of the user interface assembly 110. In FIG. 9A, the user interface assembly 110 is inserted between the buttocks of the user. For instance, with the applicator 118 in the folded state, a medical practitioner can hold the first tab 758A and the second tab 758B of the applicator 118 along with the grip regions 864 of the adhesive sheet 120. As shown in FIG. 9A, this allows the medical practitioner to hold the user interface assembly 110 at a location remote from the buttocks while positioning the aperture in the user interface 116 and the drainage conduit 112 at the anus.

In FIG. 9B, the first release liner 860 is removed from the first half of the adhesive sheet 120. For example, the medical practitioner can grasp the second end at the periphery of the user interface 116 and pull outwardly away from the user to separate the first portion 860A of the first release liner 860 from the adhesive sheet 120. In this way, the first release liner 860 can be progressively expose the first half the adhesive sheet 120 in a direction from the aperture 754 towards the peripheral edge of the user interface 116.

In FIG. 9C, after the first release liner 860 is removed from the adhesive sheet 120, the medical practitioner can use the alignment indicator 866 to determine the position of the aperture relative to the anus of the user. In some instance, the medical practitioner can use the applicator 118 to adjust a position of the user interface 116 relative to the user based on a position of the alignment indicator relative to the anus of the user.

In FIG. 9D, the first half 920A of the adhesive sheet 120 can be coupled to the skin of the user on a first buttock. Additionally, as shown in FIG. 9D, the medical practitioner can use one hand to hold the applicator 118 in position at the anus while using the other hand to place the first half 920A of the adhesive sheet 120 on the first buttock (e.g., by holding the grip region 864 of the adhesive sheet 120).

In FIG. 9E, the second release liner 960 is removed from the second half of the adhesive sheet 120. For example, the medical practitioner can grasp the second end at the periphery of the user interface 116 and pull outwardly away from the user to separate the first portion 960A of the second release liner 960 from the adhesive sheet 120. In this way, the second release liner 960 can be progressively expose the second half the adhesive sheet 120 in a direction from the aperture 754 towards the peripheral edge of the user interface 116.

In FIG. 9F, the second half 920B of the adhesive sheet 120 can be coupled to the skin of the user on a second buttock. Additionally, as shown in FIG. 9F, the medical practitioner can use one hand to hold the applicator 118 in position at the anus while using the other hand to place the second half 920B of the adhesive sheet 120 on the second buttock (e.g., by holding the grip region 864 of the adhesive sheet 120).

FIG. 9G depicts the applicator 118 being removed from the user interface 116 and FIG. 9H depicts the user interface 116 and the drainage conduit 112 after the applicator 118 has been removed from the user interface 116. For example, as shown in FIG. 9G, the first lateral portion 718A and the second lateral portion 718B can be torn apart from each other (e.g., at the first bridge portion 752A and/or the second bridge portion 752B). This can facilitate completely removing the applicator 118, which can help to improve user comfort and reduce pressure on the user's buttocks. However, in another example, the applicator 118 can be decoupled from the remainder of the user interface 116 (e.g., the adhesive sheet 120 and the collar 122, and then moved distally along the drainage conduit 112 away from the user (e.g., with the first bridge portion 752A and the second bridge portion 752B intact). FIGS. 9I-9J depicts the distal end 612B of the drainage conduit 112 being coupled to the collection reservoir 114. As shown in FIGS. 9I-9J, the collection reservoir 114 can be coupled to the side of a patient support structure (e.g., a hospital bed).

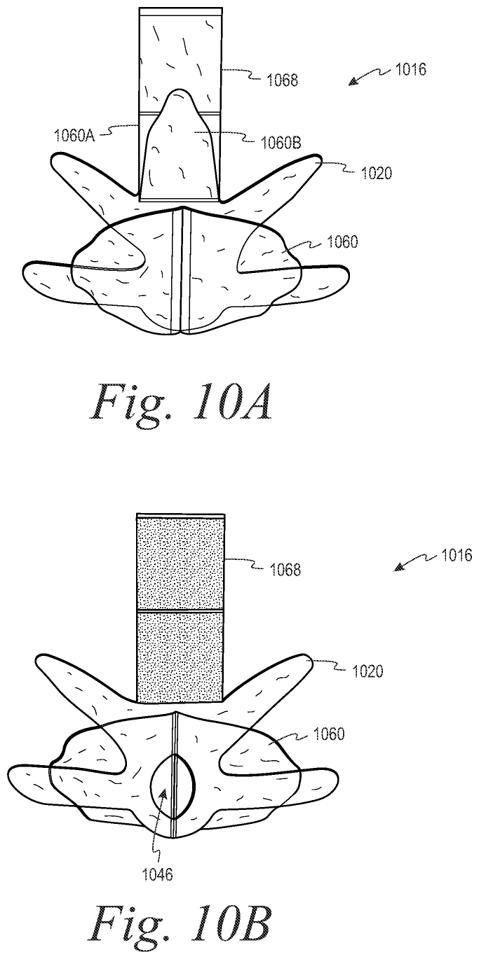

Although the applicator 118 includes the first lateral portion 318A, 618A and the second lateral portion 318B, 618B in FIGS. 3A-9J, the applicator 118 can have a different configuration in other examples. FIGS. 10A-13B depict aspects of the user interface 116 and/or the applicator 118 according to some other examples.

FIGS. 10A-10B depict a user interface 1016 for an implementation of the user interface 116 according to another example. As shown in FIG. 10A-10B, the user interface 1016 can include an adhesive sheet 1020, a release liner 1060, and an aperture 1046 in the adhesive sheet 1020. The adhesive sheet 1020 is substantially similar or identical to the adhesive sheet 120 described above, except the adhesive sheet 1020 in FIGS. 10A-10B can have a different shape. For example, in FIGS. 10A-10B, the adhesive sheet 1020 can include a plurality of arms that extend outwardly from a central portion. The central portion of the adhesive sheet 1020 can define the aperture 1046.

Additionally, the user interface 1016 includes a sacral pressure pad 1068. The sacral pressure pad 1068 can extend from a top edge of the adhesive sheet 1020 such that the sacral pressure pad 1068 is configured to be located adjacent to a sacrum of a user when the adhesive sheet 120 is coupled to the skin of the user with the aperture 1046 aligned with the anus of the user. The sacral pressure pad 1068 can have a thickness and a pliability that assists in reducing a pressure on the sacrum when the sacral pressure pad 1068 is positioned between the sacrum of the user and a support surface (e.g., a hospital bed and/or a wheel chair).

In some examples, the sacral pressure pad 1068 can include an adhesive that that is configured to couple the sacral pressure pad 1068 to the sacrum of the user. In such examples, the release liner 1060 can also include a sacral portion that covers the adhesive on the sacral pressure pad 1068 prior to adhering the sacral pressure pad 1068 to the user.

Similar to the first and second release liners described above, the sacral portion of the release liner 1060 can include a first portion 1060A and a second portion 1060B. The first portion 1060A can cover the adhesive on the sacral pressure pad 1068 to inhibit the adhesive prematurely or inadvertently adhering to an object prior to application of the user interface 116 to the user. The second portion 1060B can have a first end that is coupled to the first portion 1060A at an inner portion of the sacral pressure pad 1068 (e.g., at an end closest to the adhesive sheet 1020), and a second end that is separate from the first portion 1060A at an outer portion of the sacral pressure pad 1068 (e.g., at an end farthest away from the adhesive sheet 1020). In this arrangement, a practitioner can grasp the second end of the second portion 1060B and pull outwardly away from the user and the adhesive sheet 1020 to separate the first portion 1060A of the release liner 1060 from the adhesive on the sacral pressure pad 1068. In this way, the release liner 1060 can be progressively expose the adhesive on the sacral pressure pad 1068 in a direction from the adhesive sheet 1030 towards the peripheral edge of the user interface 116. This can help to improve coupling the adhesive to the sacrum.

FIGS. 11A-11B depict a user interface 1116 for an implementation of the user interface 116 according to another example. As shown in FIG. 11A-11B, the user interface 1116 can include an adhesive sheet 1120, a release liner 1160, an aperture 1146 in the adhesive sheet 1120, and a sacral pressure pad 1168. However, in this example, the sacral pressure pad 1168 includes a plurality of slits 1169. The slits 1169 in the sacral pressure pad 1168 can help to splay the sacral pressure pad 1168 over the sacrum of the user, and can assist in removing the sacral pressure pad 1168 from the user. These functions can be further enhanced by arranging the slits 1169 at a diagonal orientation relative to a longitudinal axis of the sacral pressure pad 1168.

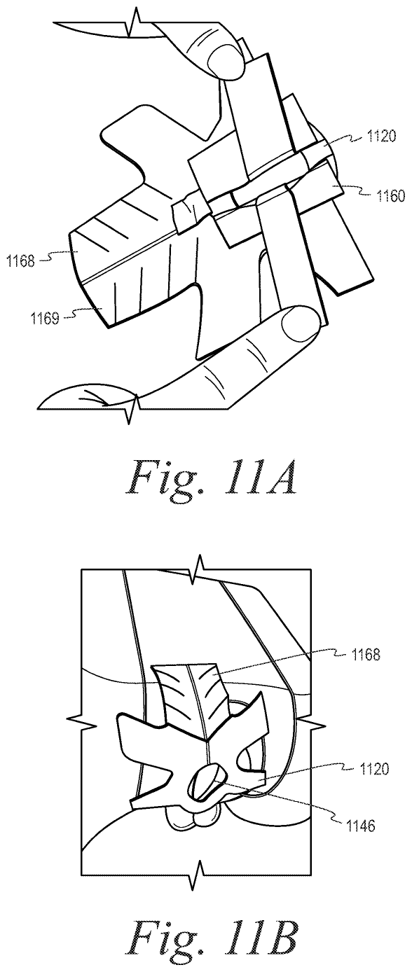

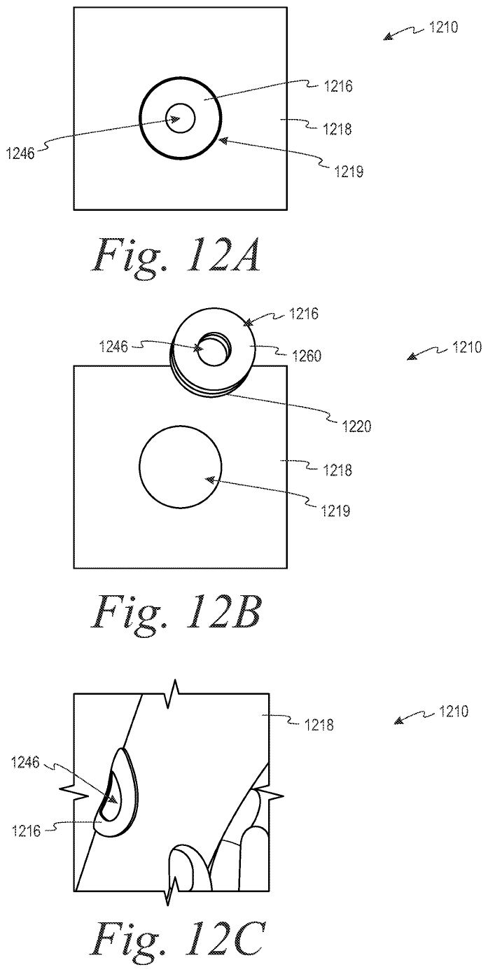

FIGS. 12A-12C depict a user interface assembly 1210 including a user interface 1216 and an applicator 1218 for an implementation of the user interface 116 and the applicator 118 and according to another example. In FIGS. 12A-12C, the user interface 1216 is disposed within an aperture 1219 in the applicator 1218 while coupling the user interface 1216 to the user, and the applicator 1218 and the user interface 1216 are configured to decouple from each other after coupling the user interface 1216 to the user.

In this example, the user interface 1216 defines an aperture 1246 that is configured to communicate with the lumen of the drainage conduit 112 described above. AS shown in FIG. 12B, the user interface 1216 can also include an adhesive sheet 1220 and a release liner 1260, as described above.

FIG. 12A and FIG. 12C depict the user interface assembly 1210 in a first configuration in which the user interface 1216 is received in the aperture 1219 of the applicator 1218. As shown in FIG. 12C, the user interface assembly 1210 is configured to be bent and/or folded along an axis extending through the user interface 1216 without the user interface 1216 decoupling from the applicator 1218. In this first configuration, an operator can hold the applicator 1218 while coupling the user interface 1216 to the user in a manner similar to that described above.

FIG. 12B depicts the user interface assembly 1210 in a second configuration in which the user interface 1216 is decoupled from the applicator 1218. In an example, the adhesive sheet 1220 can have a first breakaway force that defines an amount of force that is required to remove the user interface 1216 from the skin of the user, and the user interface assembly 1210 can have a second breakaway force that defines an amount of force that is required to decouple the applicator 1218 from the user interface 1216 along an interface between the applicator 1218 and the user interface 1216 (e.g., along a circumference of the aperture 1219). The first breakaway force can be greater than the second breakaway force. In this arrangement, when the user interface 1216 is coupled to the skin of the user, the operator can decouple and remove the applicator 1218 by pulling the applicator 1218 away from the user interface 1216 with a force that is (i) greater than the second breakaway force and (ii) less than the first breakaway force.

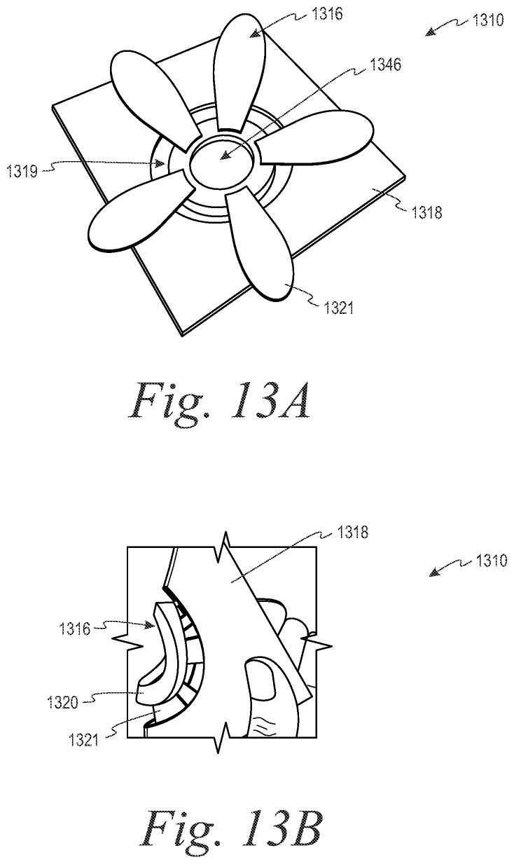

FIGS. 13A-13B depict a user interface assembly 1310 including a user interface 1316 and an applicator 1318 for an implementation of the user interface 116 and the applicator 118 and according to another example. The user interface assembly 1310 is substantially similar to the user interface assembly 1210 of FIGS. 12A-12C, except the user interface assembly 1310 includes a user interface 1316 having a plurality of arms extending from a central ring, which defines an aperture 1346 for communicating with the lumen of the drainage conduit 112.

Similar to the user interface assembly 1210 of FIGS. 12A-12C, the user interface 1316 is disposed within an aperture 1319 in the applicator 1318 while coupling the user interface 1316 to the user, and the applicator 1318 and the user interface 1316 are configured to decouple from each other after coupling the user interface 1316 to the user. However, the user interface assembly 1310 of FIGS. 13A-13B differs from the user interface assembly 1210 in that the arms of the user interface 1316 extend beyond the aperture 1346 and are coupled to the distal side of the applicator 1318 when in the first configuration. After coupling an adhesive sheet 1320 on the central ring of the user interface 1316 to the skin of a user, the applicator 1318 can be pulled away from the user interface 1316 with a force between the first breakaway force and the second breakaway force described above to decouple the applicator 1318 from the user interface 1316. In this example, while decoupling the applicator 1318 from the user interface 1316, the arms can pass through the aperture 1319 of the applicator 1318.

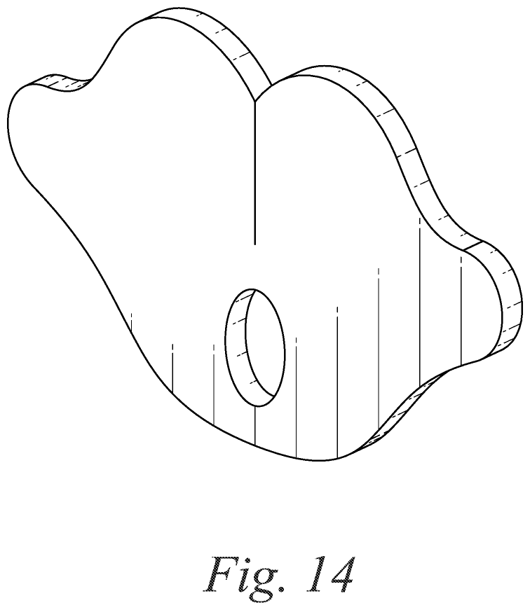

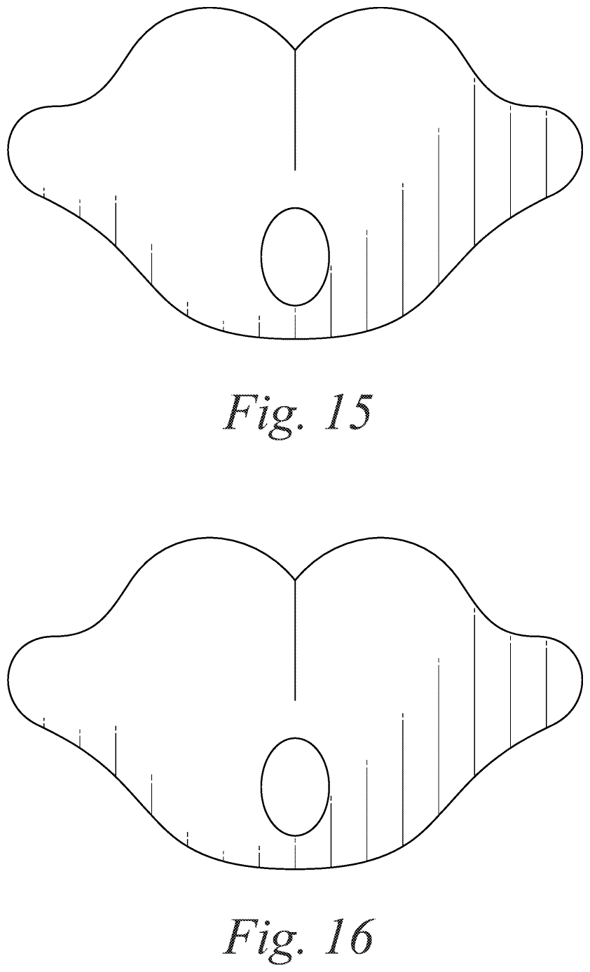

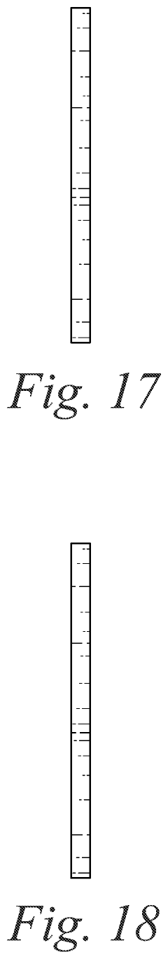

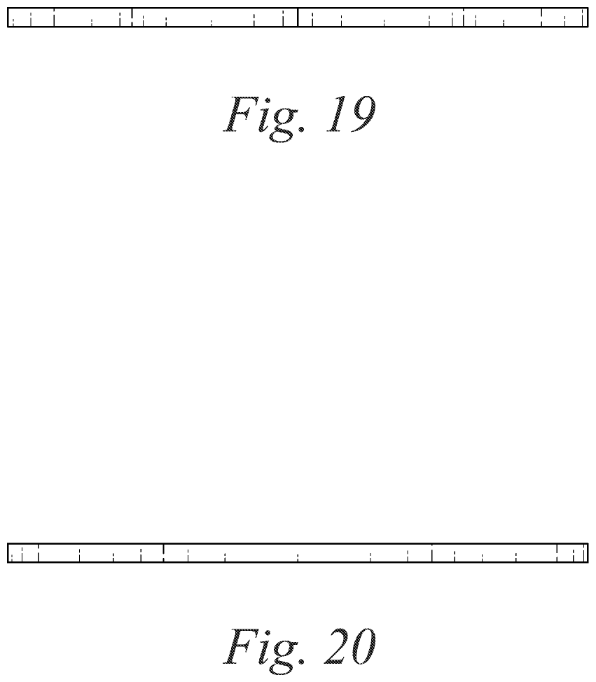

FIGS. 14-20 depict additional views of the applicator 118 shown in FIGS. 6-9G according to an example.

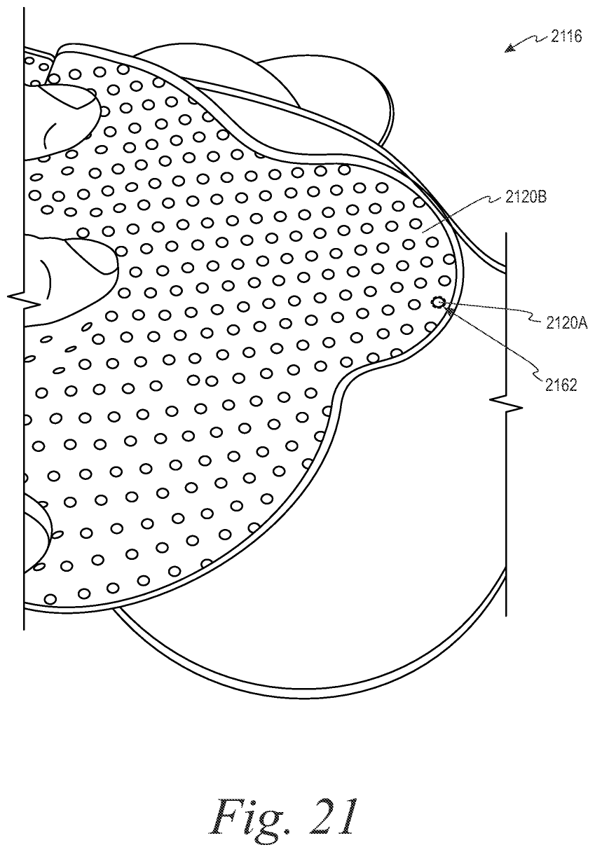

FIG. 21 depicts a user interface 2116 according to another example. The user interface 2116 shown in FIG. 21 is substantially similar or identical to the user interface 116 described above, except the user interface 2116 includes a plurality of adhesive sheets 2120A, 2021B. In particular, the adhesive sheets can include a first adhesive sheet 2120A and a second adhesive sheet 2120B. The first adhesive sheet 2120A is coupled to the proximal side of the user interface 2116, and the second adhesive sheet 2120B is coupled to a proximal side of the first adhesive sheet 21020B. As shown in FIG. 21, the second adhesive sheet 2120B includes a plurality of apertures 2162. In this arrangement, the first adhesive sheet 2120A is located between the second adhesive sheet 2120B and the user interface 2116, and portions of the first adhesive sheet 2120A are exposed through the apertures 2162 in the second adhesive sheet 2120B.

As shown in FIG. 21, the apertures can be distributed over an entirety of the second adhesive sheet 2120B. However, in other examples, a portion of the second adhesive sheet 2120b can omit the apertures. In some examples, the apertures can be uniformly distributed over one or more portions (or an entirety) of the second adhesive sheet 2120B. Although the apertures 2162 are circular in FIG. 21, the apertures 2162 can have a different shape in other examples (e.g., hexagonal shape, other polygonal shapes, and/or non-polygonal shapes).

The first adhesive sheet 2120A has a first adhesive strength, and the second adhesive sheet 2120B has a second adhesive strength, which is different than the first adhesive strength. This can help to control the overall adhesive strength applied to the skin. In one example, the first adhesive strength is greater than the second adhesive strength. In another example, the first adhesive strength is less than the second adhesive strength.

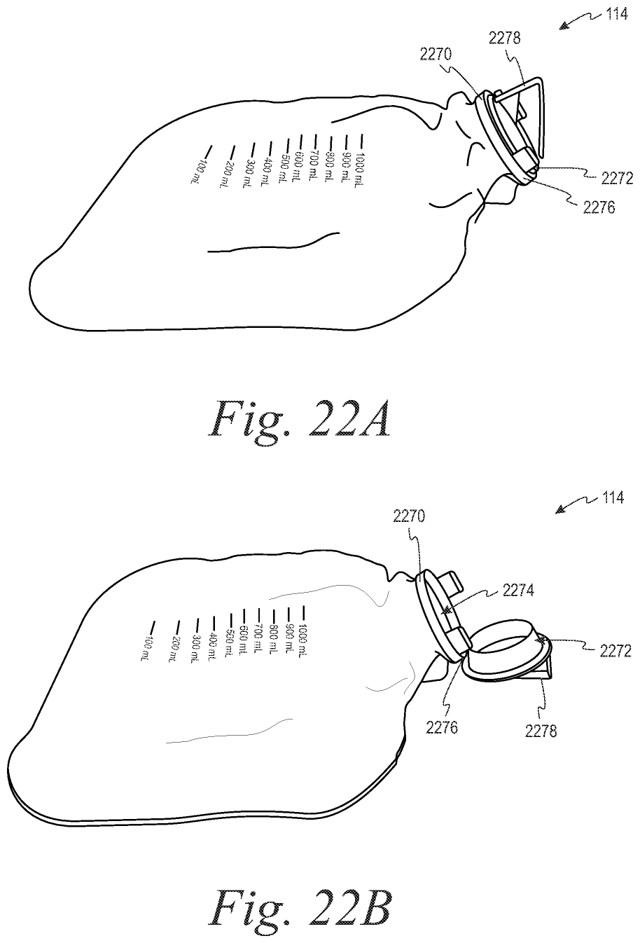

Referring now to FIGS. 22A-22B, an implementation of the collection reservoir 114 is shown according to another example. As described above, the collection reservoir 114 can be coupled to the distal end of the drainage conduit 112 (e.g., shown in FIGS. 1, 5 and 6), and the collection reservoir 114 is configured to contain the feces.

In this example, the collection reservoir 114 includes a port 2270 and a cap 2272. The port 2270 defines an opening 2274 in the collection reservoir 114 through which feces can be received from the drainage conduit 112. The cap 2272 is configured to removably couple to the port 2270 at the opening 2274. In particular, the cap 2272 is operable between (i) an open state in which the cap 2272 is decoupled from the port 2270 at the opening 2274 such the opening 2274 is uncovered by the cap 2272 (as shown in FIG. 22B), and (ii) a closed state in which the cap 2272 is coupled to the port 2270 at the opening 2274 such that the opening 2274 is covered by the cap 2272 (as shown in FIG. 22A). In this arrangement, the drainage conduit 112 can be coupled to the port 2270 to pass feces into the collection reservoir 114 when the cap 2272 is in the open state, and the cap 2272 can help to inhibit or prevent feces from leaking out of the opening 2274 of the port 2270 when the cap 2272 is in the closed state.

In FIGS. 22A-22B, the cap 2272 is coupled to an exterior surface of the port 2270 such that (i) the cap 2272 is movable between the open state and closed state, and (ii) the cap 2272 remains coupled to the port 2270 when the cap 2272 is in the open state. For instance, in FIGS. 22A-22B, the cap 2272 is coupled to the port 270 by a connector portion 2276. In one example, the connector portion 2276 can be living hinge. In another example, the connector portion 2276 can be a leash that extends between the cap 2272 and the port 2270. In some examples, the port 2270, the cap 2272, and the connector portion 2276 can be a monolithic structure. In other examples, one or more of the port 2270, the cap 2272, and the connector portion 2276 can be formed separate components that are later coupled to each other.

Additionally, in FIGS. 22A-22B, the cap 2272 includes a hook 2278. The hook 2278 can be configured to couple the collection reservoir 114 to a support structure (e.g., a hospital bed and/or wheel chair) while the cap 2272 is in the open state. This can help to hang and support the collection reservoir 114 while the drainage conduit 112 is coupled to the port 2270.

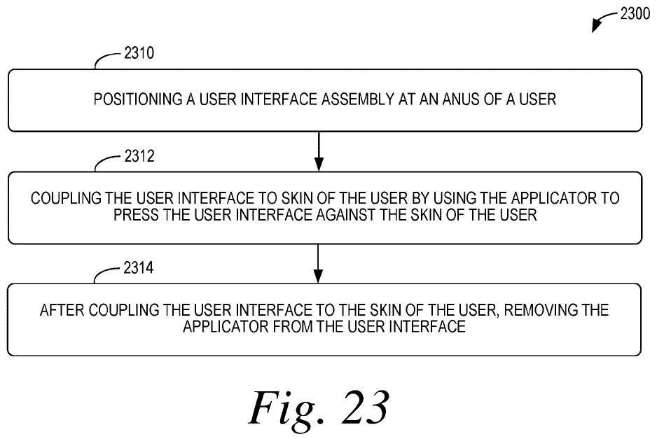

Referring now to FIG. 23, a flowchart of a process 2300 for using a fecal collection system is shown according to an example. At block 2310, the process 2300 includes positioning a user interface assembly at an anus of a user. The user interface assembly includes a user interface and an applicator. At block 2312, the process 2300 includes coupling the user interface to skin of the user by using the applicator to press the user interface against the skin of the user. At block 2314, the process 2300 includes, after coupling the user interface to the skin of the user at block 2312, removing the applicator from the user interface.

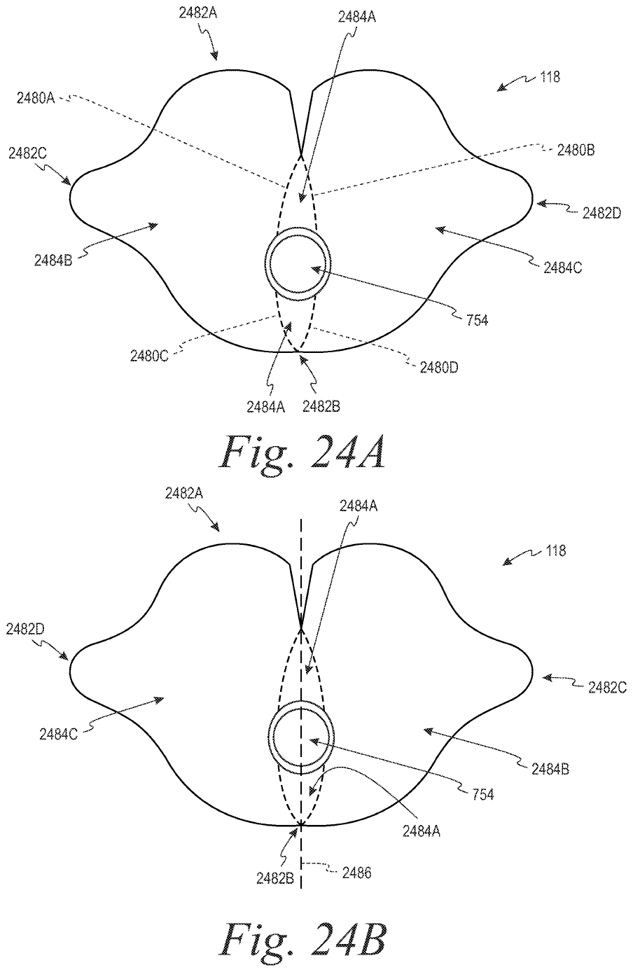

FIGS. 24A-24C show an implementation of the applicator 118, which can be used with the fecal collection systems 100, 600 described herein, according to another example. In particular, FIG. 24A shows a proximal side of the applicator 118 in the extended state, FIG. 24B shows a distal side of the applicator 118 in the extended state, and FIG. 24C shows the applicator 118 in the folded state, according to the example.

The applicator 118 is substantially similar or identical to the applicator 118 shown and described above with respect to FIGS. 1-9J and 14-20, except the applicator 118 shown in FIGS. 24A-24C includes a plurality of fold lines 2480 that are configured to form a contact surface of the applicator 118, which has a geometry that can help to improve coupling the user interface 116 to the user. For example, as described above, the applicator 118 is coupled to the distal side of the user interface 116 when assembled with the user interface (e.g., as shown in FIGS. 1 and 6-9G), the applicator 118 includes the aperture 754 extending through the proximal side and the distal side of the applicator 118 (e.g., as shown in FIGS. 24A-24C), and the aperture 754 of the applicator 118 overlaps with the aperture 646 of the user interface 116 (e.g., as shown in FIGS. 6-9G).

As shown in FIGS. 24A-24B, the applicator 118 includes a superior side 2482A, an inferior side 2482B, a first lateral side 2482C extending between the superior side 2482A and the inferior side 2482B, and a second lateral side 2482D extending between the superior side 2482A and the inferior side 2482B. In general, the superior side 2482A is configured to be positioned superior to the anus of the user when the applicator 118 is used to couple the user interface 116 to the user, the inferior side 2482B is configured to be positioned inferior to the anus of the user when the applicator is used to couple the user interface 116 to the user. The proximal side of the applicator 118 and the distal side of the applicator 118 each extend on opposing sides between the superior side 2482A, the inferior side 2482B, the first lateral side 2482C, and the second lateral side 2482D.

The applicator 118 includes a central portion 2484A extending between the superior side 2482A of the applicator 118 and an inferior side 2482B of the applicator 118, a first lateral portion 2484B on a first lateral side of the central portion 2484A, and a second lateral portion 2484C on a second lateral side of the central portion 2484A. The central portion 2484A includes the aperture 754 of the applicator 118. As shown in FIGS. 24A-24C, the applicator 118 also includes the fold lines 2480A-2480D about which the first lateral portion 2484B and the second lateral portion 2484C are configured to fold relative to the central portion 2484A.

The fold lines 2480A-2480D can thus define boundaries between the central portion 2484A and the first lateral portion 2484B, and the central portion 2484A and the second lateral portion 2484C. For example, a first subset of the fold lines 2480A, 2480C are between the central portion 2484A and the first lateral portion 2484B, and a second subset of the fold lines 2480B, 2480D are between the central portion 2484A and the second lateral portion 2484C. In this arrangement, the first lateral portion 2484B and the second lateral portion 2484C are configured to move about the first subset of the fold lines 2480A, 2480C and the second subset of the fold lines 2480B, 2480D, respectively, to actuate the applicator 118 between the extended state and the folded state. As shown in FIG. 24C, when the applicator 118 is in the folded state, the distal side of the applicator 118 at the first lateral portion 2484B faces the distal side of the applicator 118 at the second lateral portion 2484C, and the proximal side of the applicator 118 at the central portion 2484A defines one or more outwardly facing surfaces extending between the fold lines 2480A-2480D, the superior side 2482A, the inferior side 2482B, and the aperture 754.

As shown in FIGS. 24A-24C, along a width extending between the first lateral side 2482C of the of the applicator 118 and a second lateral side 2482D of the applicator 1118, the first subset of the fold lines 2480A, 2480C and the second subset of the fold lines 2480B, 2480D are separated by a non-zero distance from each other. As a result, the central portion 2484A has a width that is greater than a width of the applicator 118 shown in FIGS. 6-9G in which the first lateral portion 2484B and the second lateral portion 2484C are separated by a single fold line at the hinge portion 718C. In this arrangement, the central portion 2484A can provide a greater surface area and/or improved geometry (e.g., three-dimensional surface contours) to more accurately press the adhesive sheet 120 of the user interface 116 against the skin around the anus of the user as compared to the alternative implementations in which the applicator 118 includes a single fold line for folding the applicator 118 in half. This can help to ultimately form a better seal between the user interface 116 and the drainage conduit 112 around the anus of the user, which can help to mitigate leakage.

In the example shown in FIGS. 24A-24C, the fold lines 2480A-2480D (i) extend from the aperture 754 of the applicator 118 to the superior side 2482A of the applicator 118 and (ii) extend from the aperture 754 of the applicator 118 to the inferior side 2482B of the applicator 118. This can help to position at least a portion of the aperture 754 in the central portion 2484A when the applicator 118 is in the folded state, as shown in FIG. 24C. As a result, the central portion 2484A can provide surfaces above and below the aperture 754, whereas the first lateral portion 2484B and the second lateral portion 2484C can provide surfaces on opposing lateral sides of the aperture 754 for pressing the adhesive sheet 120 of the user interface 116 against the skin around the anus of the user.

In some examples, when the applicator 118 is in the folded state, a width of the central portion 2484A at the aperture 754 of the applicator 118 can be greater than 50 percent of the width of the aperture 754 of the applicator 118 when the applicator 118 is in the extended state. This can help to form an area of the central portion 2484A such that the applicator 118 can press the adhesive sheet 120 surrounding the drainage conduit 112 to the skin of the user surrounding the anus. In turn, this can help to provide a proper fit and obtain a seal around the anus. Additionally, with a portion of the aperture 754 at the first lateral portion 2484B and the second lateral portion 2484C, the applicator 118 can help to press the adhesive sheet 120 against a “crack” area of the user to help form the seal around the anus.

Additionally, in the example shown in FIGS. 24A-24C, a center axis 2486 of the applicator 118 (i) extends in a dimension between the superior side 2482A and the inferior side 2482B and (ii) through the aperture 754 of the applicator 118. In this example, the fold lines 2480A-2480D include a first fold line 2480A and a second fold line 2480B that (i) extend in a superior direction from opposing sides of the aperture 754 of the applicator 118 towards the superior side 2482A, and (ii) taper toward the center axis 2486 of the applicator along the superior direction. Also, in this examples, the fold lines 2480A-2480D include a third fold line 2480C and a fourth fold line 2480D that (i) extend in an inferior direction from opposing sides of the aperture 754 of the applicator 118 towards the inferior side 2482B, and (ii) taper toward the center axis 2486 of the applicator 118 along the inferior direction. This can help to provide the central portion 2482 with a concave surface profile (e.g., as shown in FIG. 24C), which can help to improve contact between the adhesive sheet 120 and the anatomy surrounding the anus of the user.

In the example shown in FIGS. 24A-24C, the fold lines 2480A-2480D can be score lines formed by a scoring machine having an edge that runs alone the applicator to create a groove or crease along the fold lines 2480A-2480D. In other examples, the fold lines 2480A-2480D additionally or alternatively can be formed by respective partial cuts that extend partially through a thickness of the applicator 118, where the thickness of the applicator 118 extends in a dimension between the proximal side and the distal side of the applicator 118. For instance, in one implementation, the respective partial cuts can each extend through approximately 75 percent of the thickness of the applicator 118. This can provide a good a balance between foldability and durability of the applicator 118. In still other examples, the fold lines 2480A-2480D can additionally or alternatively include one or more complete cuts through the thickness of the applicator 118.

In some examples, the fold lines 2480A-2480D can be formed on the distal side. In other examples, the fold lines 2480A-2480D can be formed on the proximal side. Forming the fold lines 2480A-2480D can help to enhance foldability of the applicator 118 as compared to examples in which the fold lines 2480A-2480D are formed on the distal side. In still other examples, the fold lines 2480A-2480D can be formed on a combination of the proximal side and the distal side of the applicator 118.

Additionally, the fold lines 2480A-2480D can be configured to facilitate removing the applicator 118 from the user interface 116 as described above with respect to the applicator 118 of FIG. 7. For example, terminating the fold lines 2480A-2480D at the aperture 754, tapering the fold lines 2480A-2480D towards each other, and/or forming the fold lines 2580A-2580H by partial or complete cuts can help to define the bridge portions for facilitating removal of the applicator 118 from the user interface 116 as described above.

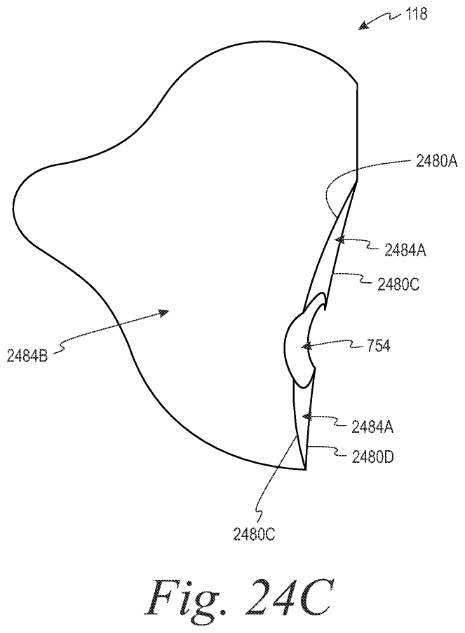

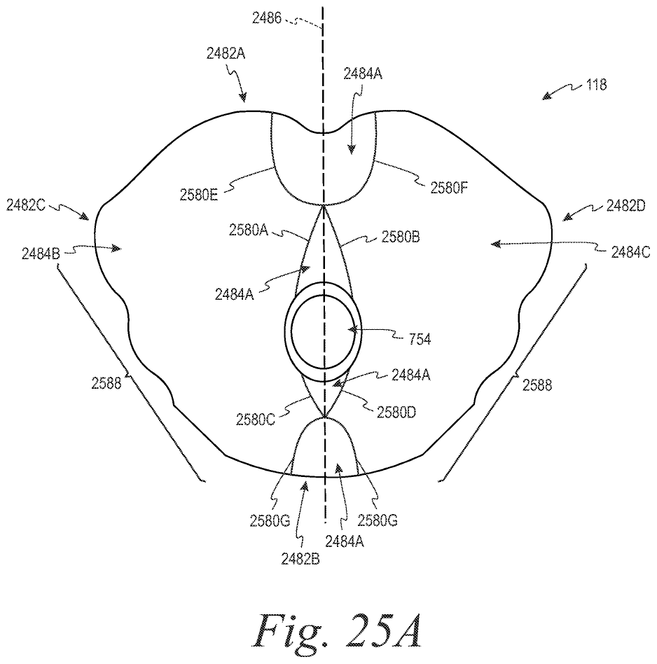

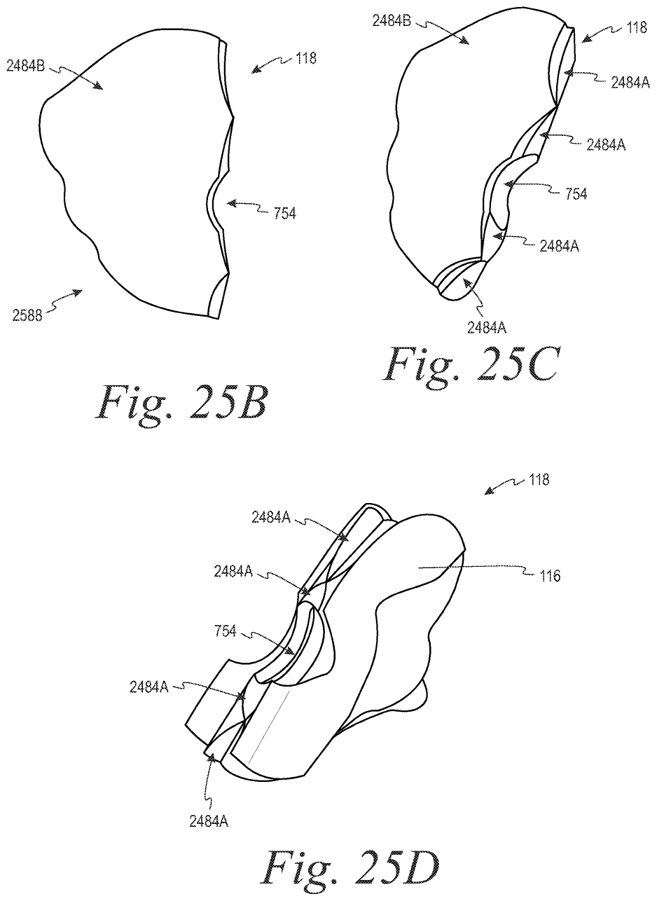

FIGS. 25A-25D show another implementation of the applicator 118, which can be used with the fecal collection systems 100, 600 described herein, according to another example. In particular, FIG. 25A shows a proximal side of the applicator 118 in the extended state, FIG. 25B shows a side view of the applicator 118 in the folded state, FIG. 25C shows a first perspective view of the applicator 118 in the folded state, and FIG. 25D shows a second perspective view of the applicator 118 in the folded state and with the user interface 116 coupled to the proximal side of the applicator 118, according to the example.

The applicator 118 is substantially similar or identical to the applicator 118 shown and described above with respect to FIGS. 24A-24C, except the fold lines have a different configuration to show another example of the three-dimensional geometries that can be provided by the central portion 2484A for assisting in coupling the user interface 116 to the skin of the user. As described above, the applicator 118 shown in FIGS. 25A-25D is coupled to the distal side of the user interface 116 when assembled with the user interface (e.g., as shown in FIGS. 1 and 6-9G), the applicator 118 includes the aperture 754 extending through the proximal side and the distal side of the applicator 118, and the aperture 754 of the applicator 118 overlaps with the aperture 646 of the user interface 116.

As shown in FIGS. 25A-25D, the applicator 118 includes the superior side 2482A, the inferior side 2482B, the first lateral side 2482C extending between the superior side 2482A and the inferior side 2482B, and the second lateral side 2482D extending between the superior side 2482A and the inferior side 2482B. The applicator 118 also includes the central portion 2484A extending between the superior side 2482A of the applicator 118 and an inferior side 2482B of the applicator 118, the first lateral portion 2484B on the first lateral side of the central portion 2484A, and the second lateral portion 2484C on the second lateral side of the central portion 2484A. The central portion 2484A includes the aperture 754 of the applicator 118.

As shown in FIGS. 25A-25D, the applicator 118 also includes a plurality of fold lines 2580A-2580H about which the first lateral portion 2484B and the second lateral portion 2484C are configured to fold relative to the central portion 2484A. The fold lines 2580A-2580H can thus define boundaries between the central portion 2484A and the first lateral portion 2484B, and the central portion 2484A and the second lateral portion 2484C. For example, a first subset of the fold lines 2580A, 2580C, 2580E, 2580G are between the central portion 2484A and the first lateral portion 2484B, and a second subset of the fold lines 2580B, 2580D, 2580F, 2580H are between the central portion 2484A and the second lateral portion 2484C. In this arrangement, the first lateral portion 2484B and the second lateral portion 2484C are configured to move about the first subset of the fold lines 2580A, 2580C, 2580E, 2580G and the second subset of the fold lines 2580B, 2580D, 2580F, 2580H, respectively, to actuate the applicator 118 between the extended state and the folded state.

In the example shown in FIGS. 25A-25D, the center axis 2486 of the applicator 118 (i) extends in a dimension between the superior side 2482A and the inferior side 2482B and (ii) through the aperture 754 of the applicator 118. In this example, the fold lines 2580A-2580H include a first fold line 2580A and a second fold line 2580B that (i) extend in a superior direction from opposing sides of the aperture 754 of the applicator 118 towards the superior side 2482A, and (ii) taper toward the center axis 2486 of the applicator along the superior direction. Also, in this examples, the fold lines 2580A-2580H include a third fold line 2580C and a fourth fold line 2580D that (i) extend in an inferior direction from opposing sides of the aperture 754 of the applicator 118 towards the inferior side 2482B, and (ii) taper toward the center axis 2486 of the applicator 118 along the inferior direction.

The fold lines 2580A-2580H can further include a fifth fold line 2580E that extends from the first fold line 2580A to the superior side 2482A and a sixth fold line 2580F that extends from the second fold line 2580B to the superior side 2482A. The fifth fold line 2580E and the sixth fold line 2580F can diverge away from the center axis 2486 of the applicator 118 along the superior direction. The fold lines 2580A-2580H can also include a seventh fold line 2580G that extends from the third fold line 2580C to the inferior side 2482B and a eighth fold line 2580H that extends from the fourth fold line 2580D to the inferior side 2482B. The seventh fold line 2580G and the eighth fold line 2580H can diverge away from the center axis 2486 of the applicator 118 along the inferior direction.

As shown in FIG. 25B-25D, when the applicator 118 is in the folded state, the distal side of the applicator 118 at the first lateral portion 2484B faces the distal side of the applicator 118 at the second lateral portion 2484C, and the proximal side of the applicator 118 at the central portion 2484A defines a plurality of outwardly facing surfaces extending between the fold lines 2580A-2580H, the superior side 2482A, the inferior side 2482B, and the aperture 754. For example, in this arrangement, the central portion 2484A includes two surfaces superior to the aperture 754 and two surfaces inferior to the aperture 754. This can help to form a plurality of contours, which may provide a better fit for certain user anatomies. Additionally, in the example shown in FIGS. 25A-25D, each of the surfaces of the central portion 2484A can have a concave shape when the applicator is in the folded state. This can help to confirm the applicator 118 and the adhesive sheet 120 to the anatomy of the user.

As shown in FIGS. 25A-25D, along a width extending between the first lateral side 2482C of the of the applicator 118 and a second lateral side 2482D of the applicator 1118, the first subset of the fold lines 2580A, 2580C, 2580E, 2580G and the second subset of the fold lines 2580B, 2580D, 2580F, 2580H are separated by a non-zero distance from each other. As a result, the central portion 2484A has a width that is greater than a width of the applicator 118 shown in FIGS. 6-9G in which the first lateral portion 2484B and the second lateral portion 2484C are separated by a single fold line at the hinge portion 718C. In this arrangement, the central portion 2484A can provide a greater surface area and/or improved geometry (e.g., three-dimensional surface contours) to more accurately press the adhesive sheet 120 of the user interface 116 against the skin around the anus of the user as compared to the alternative implementations in which the applicator 118 includes a single fold line for folding the applicator 118 in half. This can help to ultimately form a better seal between the user interface 116 and the drainage conduit 112 around the anus of the user, which can help to mitigate leakage.

In the example shown in FIGS. 25A-25D, the fold lines 2580A-2580H include partial cuts can each extend partially through the thickness of the applicator 118 (e.g., approximately 75 percent of the thickness). However, in another example, the fold lines 2580A-2580H can additionally or alternatively be score lines and/or complete cuts through the thickness of the applicator 118.

Additionally, the fold lines 2580A-2580H can be configured to facilitate removing the applicator 118 from the user interface 116 as described above with respect to the applicator 118 of FIG. 7. For example, terminating the fold lines 2580A-2580H at the aperture 754, tapering the fold lines 2580A-2580H towards each other, and/or forming the fold lines 2580A-2580H by partial or complete cuts can help to define the bridge portions for facilitating removal of the applicator 118 from the user interface 116 as described above.

Additionally, in the example shown in FIGS. 25A-25D, the first lateral side 2482C and the second lateral side 2582D of the applicator 118 can each include a plurality of ridges 2588 that are configured to indicate a grip location for holding the applicator 118 when the applicator 118 is in the folded state. In a dimension extending between the first lateral side 2482C and the second lateral side 2482D (e.g., along a width), the ridges 2588 can be positioned directly opposite the aperture 754 when the applicator 118 is in the folded state. This can help the medical practitioner grip the applicator 118 at a positions that can assist in using the applicator 118 to press the user interface 116 with a suitable distribution of force to the skin around the anus of the user.

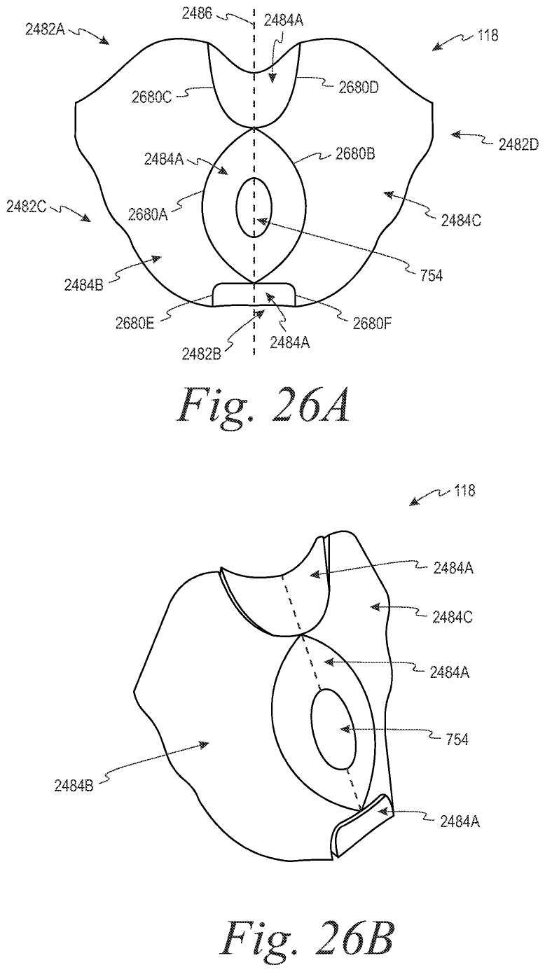

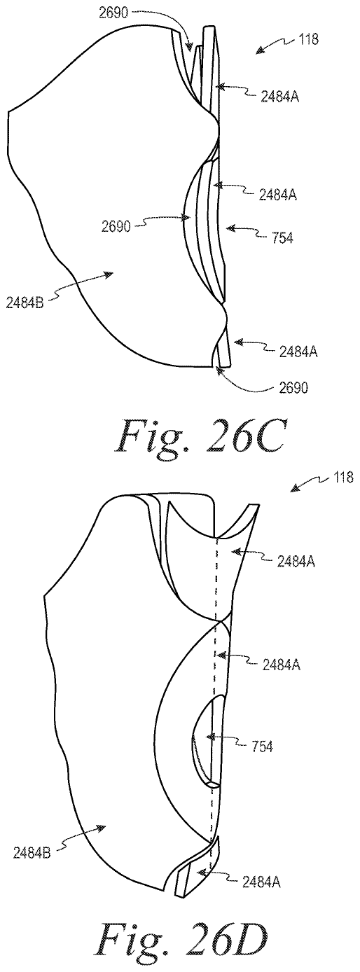

FIGS. 26A-26D show another implementation of the applicator 118, which can be used with the fecal collection systems 100, 600 described herein, according to another example. In particular, FIG. 26A shows a proximal side of the applicator 118 in the extended state, FIG. 26B shows a perspective view of the applicator 118 in a partially folded state between the extended state and the folded state, FIG. 26C shows a side view of the applicator 118 in the folded state, and FIG. 26D shows a perspective view of the applicator 118 in the folded state, according to the example.

The applicator 118 is substantially similar or identical to the applicator 118 shown and described above with respect to FIGS. 25A-25D, except the fold lines have a different configuration to show another example of the three-dimensional geometries that can be provided by the central portion 2484A for assisting in coupling the user interface 116 to the skin of the user. As described above, the applicator 118 shown in FIGS. 26A-26D is coupled to the distal side of the user interface 116 when assembled with the user interface (e.g., as shown in FIGS. 1 and 6-9G), the applicator 118 includes the aperture 754 extending through the proximal side and the distal side of the applicator 118, and the aperture 754 of the applicator 118 overlaps with the aperture 646 of the user interface 116.

As shown in FIGS. 26A-26D, the applicator 118 includes the superior side 2482A, the inferior side 2482B, the first lateral side 2482C extending between the superior side 2482A and the inferior side 2482B, and the second lateral side 2482D extending between the superior side 2482A and the inferior side 2482B. The applicator 118 also includes the central portion 2484A extending between the superior side 2482A of the applicator 118 and an inferior side 2482B of the applicator 118, the first lateral portion 2484B on the first lateral side of the central portion 2484A, and the second lateral portion 2484C on the second lateral side of the central portion 2484A. The central portion 2484A includes the aperture 754 of the applicator 118.

As shown in FIGS. 26A-26D, the applicator 118 also includes a plurality of fold lines 2680A-2680F about which the first lateral portion 2484B and the second lateral portion 2484C are configured to fold relative to the central portion 2484A. The fold lines 2680A-2680F can thus define boundaries between the central portion 2484A and the first lateral portion 2484B, and the central portion 2484A and the second lateral portion 2484C. For example, a first subset of the fold lines 2680A, 2680C, 2680E are between the central portion 2484A and the first lateral portion 2484B, and a second subset of the fold lines 2680B, 2680D, 2680F are between the central portion 2484A and the second lateral portion 2484C. In this arrangement, the first lateral portion 2484B and the second lateral portion 2484C are configured to move about the first subset of the fold lines 2680A, 2680C, 2680E and the second subset of the fold lines 2680B, 2680D, 2680F, respectively, to actuate the applicator 118 between the extended state and the folded state.

In FIGS. 26A-26D, the fold lines 2680A-2680F extend entirely around the aperture 754 of the applicator 118 such that the aperture 754 is entirely positioned within the central portion 2484A. For instance, in the example shown in FIGS. 26A-26D, the center axis 2486 of the applicator 118 (i) extends in a dimension between the superior side 2482A and the inferior side 2482B and (ii) through the aperture 754 of the applicator 118. In this example, the fold lines 2680A-2680F include a first fold line 2680A and a second fold line 2680B that extend around the aperture 754. For example, the first fold line 2680A and the second fold line 2680B can define an ovular shape and/or an elliptical shape around the aperture 754. As shown in FIGS. 26B-26D, this results in the aperture 754 being entirely positioned within the central portion 2484A. This can help to concentrate a force applied by the applicator 118 to the user around the anus of the user.

Also, in this examples, the fold lines 2680A-2680F can include a third fold line 2680C that extends from the first fold line 2680A to the superior side 2482A and a fourth fold line 2680D that extends from the second fold line 2580B to the superior side 2482A. The third fold line 2680C and the fourth fold line 2680D can diverge away from the center axis 2486 of the applicator 118 along the superior direction. The fold lines 2680A-2680F can also include a fifth fold line 2680E that extends from the first fold line 2680A to the inferior side 2482B and a sixth fold line 2680F that extends from the second fold line 2680B to the inferior side 2482B. The fifth fold line 2680E and the sixth fold line 2680F can diverge away from the center axis 2486 of the applicator 118 along the inferior direction.

As shown in FIG. 26B-26D, when the applicator 118 is in the folded state, the distal side of the applicator 118 at the first lateral portion 2484B faces the distal side of the applicator 118 at the second lateral portion 2484C, and the proximal side of the applicator 118 at the central portion 2484A defines a plurality of outwardly facing surfaces extending between the fold lines 2680A-2680F, the superior side 2482A, and the inferior side 2482B. For example, in this arrangement, the central portion 2484A includes three surfaces (e.g., a first surface surrounding the aperture, second surface superior to the first surface, and a third surface inferior to the first surface). This can help to provide a better fit for certain anatomies of the user.

As shown in FIGS. 25A-25D, along a width extending between the first lateral side 2482C of the of the applicator 118 and a second lateral side 2482D of the applicator 1118, the first subset of the fold lines 2680A, 2680C, 2680E and the second subset of the fold lines 2680B, 2680D, 2680F are separated by a non-zero distance from each other. As a result, the central portion 2484A has a width that is greater than a width of the applicator 118 shown in FIGS. 6-9G in which the first lateral portion 2484B and the second lateral portion 2484C are separated by a single fold line at the hinge portion 718C. In this arrangement, the central portion 2484A can provide a greater surface area and/or improved geometry (e.g., three-dimensional surface contours) to more accurately press the adhesive sheet 120 of the user interface 116 against the skin around the anus of the user as compared to the alternative implementations in which the applicator 118 includes a single fold line for folding the applicator 118 in half. This can help to ultimately form a better seal between the user interface 116 and the drainage conduit 112 around the anus of the user, which can help to mitigate leakage.

In the example shown in FIGS. 26A-26D, the fold lines 2680A-2680F include respective complete cuts that each extend entirely through the thickness of the applicator 118. As a result the applicator 118 can include one or more gaps 2690 between the central portion 2484A and the first lateral portion 2484B, and between the central portion 2484A and the second lateral portion 2484C. The fold lines 2680A-2680F can intersect at respective interfaces that omit the complete cuts (e.g., are not cut at all, are partially cut, or are scored). As shown in FIGS. 26B-26D, this results in the central portion 2484A having surfaces that substantially planar or convex. This can help to conform to an anatomy of the user when the applicator 118 is pressed against it.

Additionally, the fold lines 2680A-2680F can be configured to facilitate removing the applicator 118 from the user interface 116 as described above with respect to the applicator 118 of FIG. 7. For example, in FIGS. 26A-26D, tapering the fold lines 2680A-2680F towards each other and/or forming the fold lines 2580A-2580H by complete cuts can help to define the bridge portions for facilitating removal of the applicator 118 from the user interface 116 as described above.

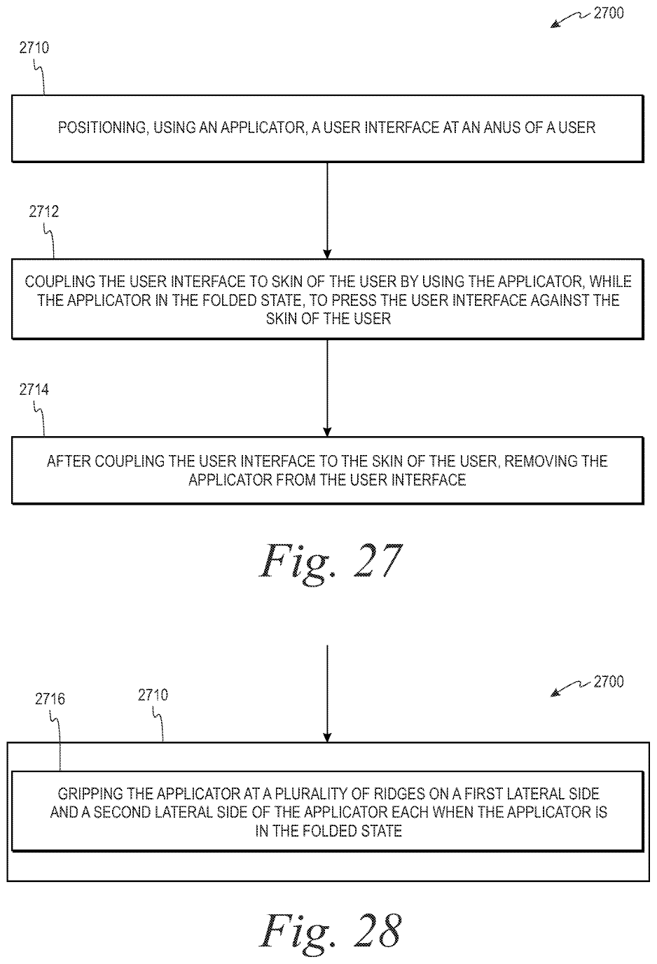

Referring now to FIG. 27, a flowchart of a process 2700 for using a fecal collection system is shown according to an example. At block 2710, the process 2700 includes positioning, using an applicator, a user interface at an anus of a user. The user interface has a proximal side and a distal side. The proximal side is configured to couple to skin of a user. The user interface includes an aperture extending through the proximal side and the distal side. The applicator is coupled to the distal side of the user interface. The applicator includes an aperture extending through a proximal side and a distal side of the applicator.

The applicator further includes a central portion extending between a superior side of the applicator and an inferior side of the applicator, a first lateral portion on a first lateral side of the central portion, and a second lateral portion on a second lateral side of the central portion. The central portion includes the aperture of the applicator. The applicator also includes a plurality of fold lines about which the first lateral portion and the second lateral portion are configured to fold relative to the central portion. A first subset of the plurality of fold lines are between the central portion and the first lateral portion. A second subset of the plurality of fold lines are between the central portion and the second lateral portion. Along a width extending between a first lateral side of the of the applicator and a second lateral side of the applicator, the first subset of the plurality of fold lines and the second subset of the plurality of fold lines are separated by a non-zero distance from each other. The first lateral portion and the second lateral portion are configured to move about the first subset of the plurality of fold lines and the second subset of the plurality of fold lines, respectively, to actuate the applicator between an extended state and a folded state.

At block 2712, the process 2700 includes coupling the user interface to skin of the user by using the applicator, while the applicator in the folded state, to press the user interface against the skin of the user. After coupling the user interface to the skin of the user at block 2712, the process 2700 includes removing the applicator from the user interface at block 2714.

FIGS. 28-30 depict additional aspects of the process 2700 according to further examples. As shown in FIG. 28, positioning the user interface at the anus of the user at block 2710 can include gripping the applicator at a plurality of ridges on a first lateral side and a second lateral side of the applicator each when the applicator is in the folded state at block 2716.

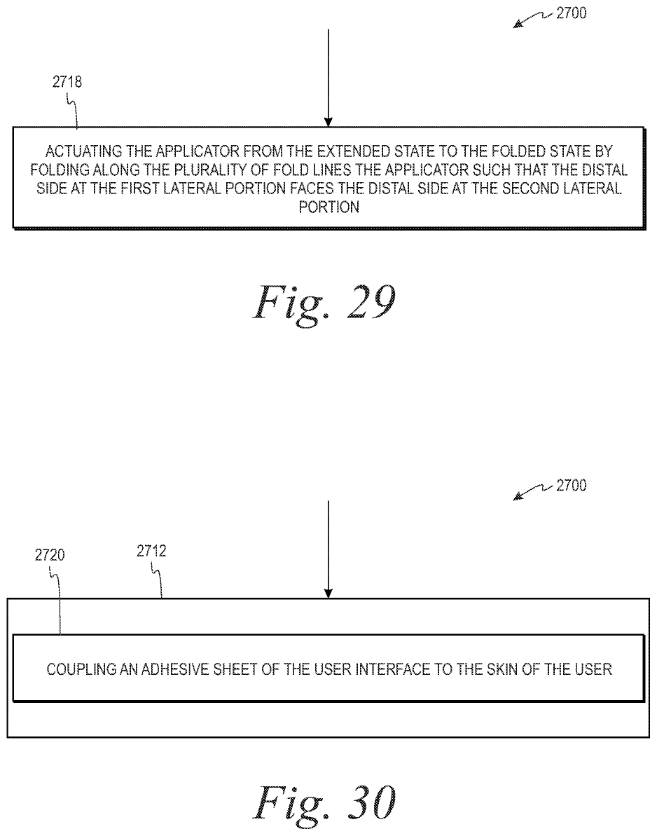

As shown in FIG. 29, the process 2700 can also include actuating the applicator from the extended state to the folded state by folding along the plurality of fold lines the applicator such that the distal side at the first lateral portion faces the distal side at the second lateral portion at block 2718.

As shown in FIG. 30, coupling the user interface to skin of the user at block 2712 can include coupling an adhesive sheet of the user interface to the skin of the user at block 2720.

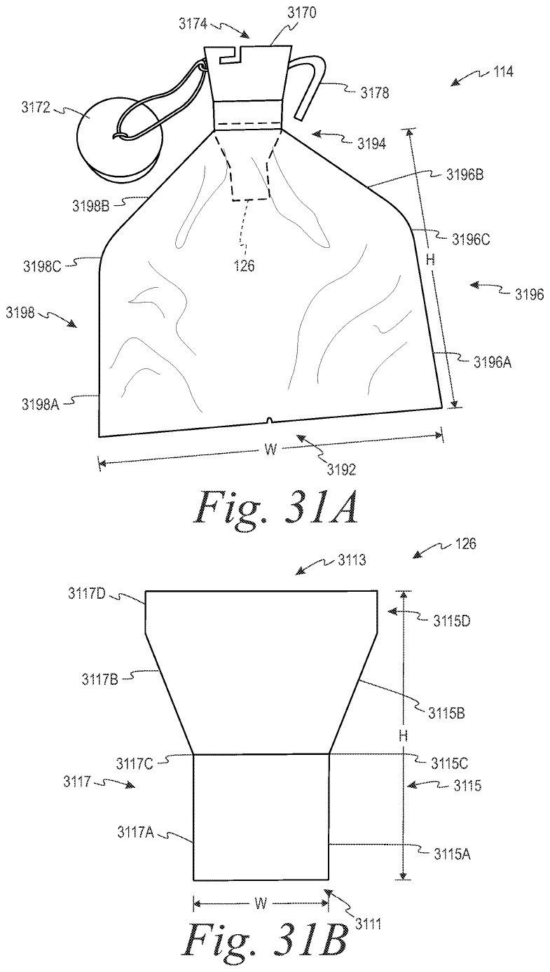

Referring now to FIG. 31A, an implementation of the collection reservoir 114 is shown according to another example. As described above, the collection reservoir 114 can be coupled to the distal end of the drainage conduit 112 (e.g., shown in FIGS. 1, 5, 6, and 22A-22B), and the collection reservoir 114 is configured to contain the feces. The implementation of the collection reservoir 114 shown in FIG. 31A can be substantially similar or identical the collection reservoir 114 described above with respect to the other Figures, except the collection reservoir 114 shown in FIG. 31A has a shape that can help to promote consistent filling and/or provide an indicator as to when the collection reservoir 114 is full.

As shown in FIG. 31A, the collection reservoir 3114 can include a distal end 3192, a proximal end 3194, a first lateral side 3196 that extends between the distal end 3192 and the proximal end 3194, and a second lateral side 3198 that extends between the distal end 319 and the proximal end 3194 and is opposite the first lateral side 3196. The collection reservoir 114 can also include a first surface and a second surface on opposing sides of the collection reservoir 114, and extending between the distal end 3192, the proximal end 3194, the first lateral side 3196, and the second lateral side 3198.

The proximal end 3194 can define an aperture for receiving the feces into the collection reservoir 114. The distal end 3192, the first lateral side 3196, the second lateral side 3198, the first surface, and the second surface can define an interior cavity for receiving and containing feces received from the drainage conduit 112. For instance, the collection reservoir 114 can be made from a fluid impermeable material (e.g., silicone, rubber, latex, and/or a thermoplastic). In one implementation, for example, the first surface and the second surface can be respective sheets of fluid impermeable material that can be coupled to each other by, for instance, radio frequency (RF) heat sealing and/or RF welding. In some implementations of such an example, the distal end 3192, the first lateral side 3196, and the second lateral side 3198 can be respective interfaces at which the sheets of the fluid impermeable material are coupled to each other. In other examples, the distal end 3192, the first lateral side 3196, and/or the second lateral side 3198 can be defined by respective sheets of the fluid impermeable material that are distinct from the sheets of fluid impermeable material that define the first surface and/or the second surface of the collection reservoir 114.

As shown in FIG. 31A, the first lateral side 3196 can include a first distal portion 3196A, a first proximal portion 3196B, and a first corner 3196C at an interface between the first distal portion 3196A and the first proximal portion 3196B. The second lateral side 3198 can include a second distal portion 3198A, a second proximal portion 3198B, and a second corner 3198C at an interface between the second distal portion 3198A and the second proximal portion 3198B. The first corner 3196C and the second corner 3198C can be configured such that the first proximal portion 3196B and the second proximal portion 3198B taper inwardly from the first distal portion 3196A and the second distal portion 3198A, respectively, along a proximal direction towards the proximal end 3194. As such, a width W of the collection reservoir 114 (e.g., in a dimension extending between the first lateral side 3196 and a second lateral side 3198) can reduce in size along the proximal direction from the first corner 3169C and the second corner 3198C towards the proximal end 3194.

In this arrangement, the first corner 3196C and the second corner 3196D can provide a visual indicator of a fill line to a practitioner such that a practitioner can determine that the collection reservoir 114 has been filled with a predetermined amount of feces (and should be empty or replaced with another collection reservoir 114). For instance, when feces enters the internal cavity of the collection reservoir 114 through the aperture at the proximal end 3194, the feces can be move into a distal portion of the collection reservoir 114 that is distal of the first corner 3196C and the second corner 3198C (e.g., the distal portion being defined by the first distal portion 3196A, the second distal portion 3198A, the distal end 3192, the first surface, and the second surface). The tapered shape of the first proximal portion 3196B and the second proximal portion 3198B can help to promote movement of feces towards the distal portion. When the predetermined amount of feces has been received in the collection reservoir 114, the feces will fill the internal cavity of the collection reservoir 114 up to the first corner 3196C and/or the second corner 3198C. Responsive to the feces filling the internal cavity up to the first corner 3196C and/or the second corner 3198C, a determination can be made that the collection reservoir 114 is full and should be emptied and/or replaced with another collection reservoir 114.

In some examples, the width W of the collection reservoir 114 can be constant over a height H of the first distal portion 3196A and the second distal portion 3198A. This can help to make the first corner 3196C and the second corner 3196D easily identifiable. However, in other examples, the width W of the collection reservoir can vary over the height H of the first distal portion 3196A and the second distal portion 3198A.

In some examples, a ratio of (i) the height H of the first distal portion 3196A and the second distal portion 3198A relative to (ii) the height H of the first proximal portion 3196B and the second proximal portion 3198B can be approximately 5:4. For instance, in the implementation shown in FIG. 31A, the height H of the first distal portion 3196A and the second distal portion 3198A can be approximately 5 inches and the height H of the first proximal portion 3196B and the second proximal portion 3198B can be approximately 4 inches. Additionally, in FIG. 31A, the width W of the collection reservoir 114 is approximately 9 inches at the distal end 3192, and the width W of the collection reservoir is approximately 3.1 inches at the proximal end 3194. However, the collection reservoir 114 can have different dimensions in other examples.

In FIG. 31A, an interface between the distal end 3192 and the first distal portion 3196A and an interface between the distal end 3192 and the second distal portion 3198A can define an angle of approximately 90 degrees. However, in other examples, these interfaces can define other angles and/or the interfaces can be rounded.

Additionally, in FIG. 31A, the collection reservoir 114 includes a port 3170 and a cap 3172. The port 3170 defines an opening 3174 in the collection reservoir 114 through which feces can be received from the drainage conduit 112. For instance, the sheets of fluid impermeable material can be coupled to the port 3170 such that the opening 3174 is in communication with the aperture at the proximal end 3194.

The cap 3172 is configured to removably couple to the port 3170 at the opening 3174. In particular, the cap 3172 is operable between (i) an open state in which the cap 3172 is decoupled from the port 3170 at the opening 3174 such the opening 3174 is uncovered by the cap 3172 (as shown in FIG. 31A), and (ii) a closed state in which the cap 3172 is coupled to the port 3170 at the opening 3174 such that the opening 3174 is covered by the cap 3172 (e.g., in a manner similar to that shown in FIG. 22A). In this arrangement, the drainage conduit 112 can be coupled to the port 3170 to pass feces into the collection reservoir 114 when the cap 3172 is in the open state, and the cap 3172 can help to inhibit or prevent feces from leaking out of the opening 3174 of the port 3170 when the cap 3172 is in the closed state.

In FIG. 31A, the cap 3172 can be coupled to an exterior surface of the port 3170 such that (i) the cap 3172 is movable between the open state and closed state, and (ii) the cap 3172 remains coupled to the port 3170 when the cap 3172 is in the open state. For instance, in FIG. 31A, the cap 3172 is coupled to the port 3170 by a connector portion 3176 in the form of a leash. This can help to facilitate coupling the cap 3172 to the port 3170 by a bayonet-type coupling in FIG. 31A. However, in other examples, the connector portion 3176 can be living hinge as described above with respect to FIGS. 22A-22B.

Additionally, in FIG. 31A, the port 3170 includes a hook 3178. The hook 3178 can be configured to couple the collection reservoir 114 to a support structure (e.g., a hospital bed and/or wheel chair) while the cap 3172 is in the open state or the closed state. This can help to hang and support the collection reservoir 114 while the drainage conduit 112 is coupled to the port 3170. In other examples, the hook 3178 can be included in the cap 3172 as described above.

As described above, the collection reservoir 114 can include a second one-way valve 126 that is configured to allow feces to flow from the drainage conduit 112 to the collection reservoir 114 and inhibit the feces from flowing from the collection reservoir 114 to the drainage conduit 112. This can also help inhibit feces from migrating back toward and into contact with the skin of the user. In one example, the drainage conduit 112 can have a diameter of approximately 1 inch to approximately 3 inches at the distal end 212B.

As shown in FIG. 31A, the second one-way valve 126 extends from the proximal end 3194 (and/or the port 3170) proximally into the internal cavity of the collection reservoir 114. FIG. 31B depicts an enlarged view of the second one-way valve 126 of the collection reservoir 114 shown in FIG. 31A.

As shown in FIGS. 31A-31B, the second one-way valve 126 includes distal end 3111, a proximal end 3113, a first lateral side 3115 that extends between the distal end 3111 and the proximal end 3113, and a second lateral side 3117 that extends between the distal end 3111 and the proximal end 3113 and is opposite the first lateral side 3115. The second one-way valve 126 can also include a first surface and a second surface on opposing sides of the second one-way valve 126, and extending between the distal end 3111, the proximal end 3113, the first lateral side 3115, and the second lateral side 3117.

The distal end 3111 and the proximal end 3113 can define apertures for receiving the feces through the second one-way valve 126 and into the collection reservoir 114, respectively. The first lateral side 3115, the second lateral side 3117, the first surface, and the second surface can define a passage for passing feces received from the drainage conduit 112 into the collection reservoir 114, and inhibiting feces from returning from the internal cavity of the collection reservoir 114 back through the proximal end 3194 and/or the port 3170.

Within examples, the second one-way valve 126 can be made from a fluid impermeable material (e.g., silicone, rubber, latex, and/or a thermoplastic). In one implementation, for example, the first surface and the second surface can be respective sheets of fluid impermeable material that can be coupled to each other by, for instance, radio frequency (RF) heat sealing and/or RF welding. In some implementations of such an example, the first lateral side 3115 and the second lateral side 3117 can be respective interfaces at which the sheets of the fluid impermeable material are coupled to each other.

The proximal end 3113 of the second one-way valve 126 is coupled to the proximal end 3194 of the collection reservoir 114 and/or the port 3170 such that the opening at the proximal end 3133 of the second one-way valve is always open (e.g., the sheets of fluid impermeable material are held in a spaced-apart arrangement). By contrast, the distal end 3111 of the second one-way valve 126 is disposed freely in the internal cavity of the collection reservoir 114. In this arrangement, the distal end 3111 collapses on itself when no feces is passing through the passage of the second one-way valve 126 in the distal direction from the distal end 3111 towards the proximal end 3113. In this way, the distal end 3111 can inhibit backflow of feces in the proximal direction while allowing feces to move in the distal direction from the drainage conduit 112 into the collection reservoir 114.

As shown in FIGS. 31A-31B, the first lateral side 3115 can include a first distal portion 3115A, a first proximal portion 3115B, and a first corner 3115C at an interface between the first distal portion 3115A and the first proximal portion 3115B. The second lateral side 3117 can include a second distal portion 3117A, a second proximal portion 3117B, and a second corner 3117C at an interface between the second distal portion 3117A and the second proximal portion 3117B. The first corner 3115C and the second corner 3117C can be configured such that the first distal portion 3115A and the second distal portion 3117A taper inwardly from the first proximal portion 3115B and the second proximal portion 3117B, respectively, along a proximal direction towards the distal end 3111. As such, a width W of the second one-way valve 126 (e.g., in a dimension extending between the first lateral side 3115 and a second lateral side 3117) can reduce in size along the distal direction from the first corner 3115C and the second corner 3117C towards the distal end 3111. The relatively narrower width of the distal end 3111 relative to the proximal end 3113 can further help to inhibit backflow of feces.

Additionally, in some examples, the first lateral side 3115 can include a first non-tapered section 3115D that is between the first proximal portion 3115B and the proximal end 3113, and the second lateral side 3117 can include a second non-tapered section 3117D that is between the second proximal portion 3117B and the proximal end 3113. The first non-tapered section 3115D and the second non-tapered section 3117D can help to facilitate coupling the second one-way valve 126 to the proximal end 3194 and/or the port 3170.

As shown in FIG. 31A, the distal end 3111 of the second one-way valve 126 can be positioned proximally of the first corner 3196C and the second corner 3198C of the collection reservoir 114. As such, when the collection reservoir 114 is filled with feces up to the fill line indicated by the first corner 3196C and the second corner 3198C, the distal end 3111 of the second one-way valve 126 is spaced away from the feces. This can further help to inhibit backflow of the feces from the internal cavity of the collection reservoir 114 through the proximal end 3194 and/or the port 3170.

In one example, the width W of the second one-way valve 126 at the distal end 3111 can be approximately 1.75 inches, and the width W of the second one-way valve 126 at the proximal end 3113 can be approximately 3.1 inches. Also, in this example, the height H of the first distal portion 3115A and the second distal portion 3117A can be approximately 1.5 inches, the height of the first proximal portion 3115B and the second proximal portion 3117B can be approximately 1.5 inches, and the height H of the first non-tapered section 3115D and the second non-tapered section 3117D can be approximately 0.5 inches. Thus, in this example, the height H of the second one-way valve 126 can be approximately 3.5 inches. The second one-way valve 126 can have other dimensions.

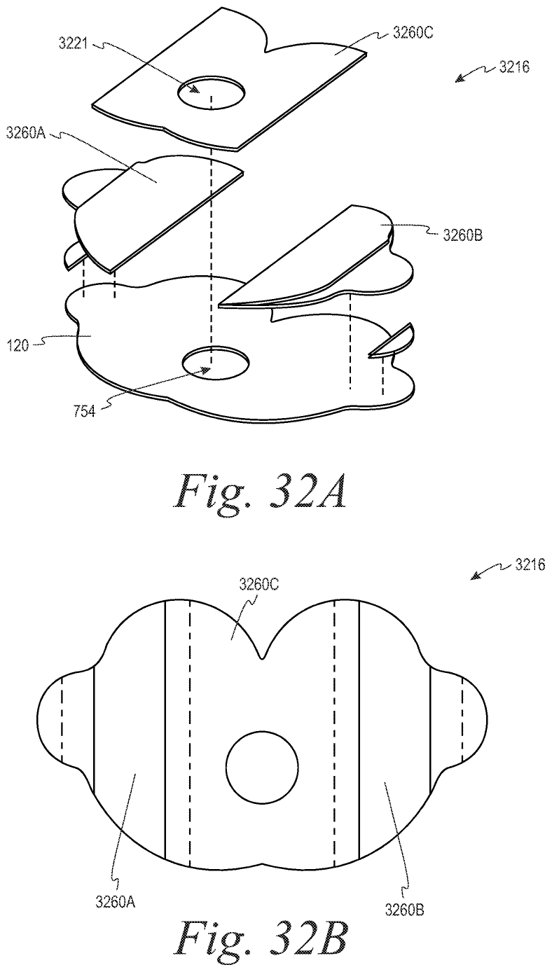

FIGS. 32A-32B depict a user interface 3216 according to another example. The user interface 3216 shown in FIGS. 32A-32B is substantially similar or identical to the user interface 116, 2116 described above, except the user interface 3216 includes an arrangement for a release liner 3260 according to another example that can be used with any of the user interfaces described herein. FIG. 32A depicts an exploded view of the user interface 3216, and FIG. 32B depicts a view of a proximal side of the user interface 3216, according to the example.

As described above, the user interface 3216 can include one or more adhesive sheets 120 on the proximal side of the user interface 3216 (e.g., the first adhesive sheet 2120A and/or the second adhesive sheet 2120B described above with respect to FIG. 21). Additionally, the user interface 3216 includes a first lateral release liner 3260A, a second lateral release liner 3260B, and a middle release liner 3260C.

The middle release liner 3260C is removably coupled to a middle portion of the adhesive sheet(s) 120, which includes the aperture 754. As shown in FIG. 32A, the middle release liner 3260C can include a liner aperture 3221 that is aligned with the aperture 754 of the adhesive sheet(s) 120. This can help to visualize the alignment of aperture 754 of the adhesive sheet(s) 120 from the proximal side.

The first lateral release liner 3260A is removably coupled to a first lateral portion of the adhesive sheet(s) 120 (e.g., on a first side of the aperture 754 and at least partially on the first side of the middle release liner 3260C), and the second lateral release liner 3260B is removably coupled to a second lateral portion of the adhesive sheet(s) 120 (e.g., on a second side of the aperture 754 and at least partially on the second side of the middle release liner 3260C). As shown in FIG. 32B, the middle release liner 3260C is removably coupled to the adhesive sheet(s) 120 between the first lateral release liner 3260A and the second lateral release liner 3260B.

In this arrangement, the user interface 3216 can be coupled to the user's skin by first removing the middle release liner 3260C from the adhesive sheet(s) 120 while the first lateral release liner 3260A and the second lateral release liner 3260B remain coupled to the adhesive sheet(s) 120. After removing the middle release liner 3260C, the adhesive sheet(s) 120 are coupled to the user's skin by a middle portion of the adhesive sheet(s) 120 that is exposed while the middle release liner 3260C is removed, and the first lateral release liner 3260A and the second lateral release liner 3260B covers lateral portions of the adhesive sheet(s) 120. Next, the first lateral release liner 3260A and the second lateral release liner 3260B liner can be independently removed from the adhesive sheet(s) 120 to provide for coupling the user interface 116 to the user's skin on one lateral portion at a time. This arrangement and sequence of operation can help to more accurately position the user interface 3216 on the user's skin and/or can help to smooth the adhesive onto the user's anatomy.

In FIGS. 32A-32B, the first lateral release liner 3260A and the second lateral release liner 3260B can each include first and second portions (e.g., as described above for the first portion 860A and the second portion 860B). In this arrangement, a practitioner can grasp the second end at the periphery of the user interface 116 and pull outwardly away from the user to separate the first portion of the first lateral release liner 3260A from the adhesive sheet(s) 120 and progressively expose the first lateral portion the adhesive sheet(s) 120 in a direction from the aperture 754 towards the peripheral edge of the user interface 116. Similarly, the practitioner can grasp the second end at the periphery of the user interface 116 and pull outwardly away from the user to separate the first portion of the second lateral release liner 3260B from the adhesive sheet(s) 120 to progressively expose the second lateral portion of the adhesive sheet(s) 120 in a direction from the aperture 754 towards the peripheral edge of the user interface 116. This can help to improve the coupling the adhesive sheet 120 to the buttocks of the user with the aperture 754 aligned with the anus of the user.

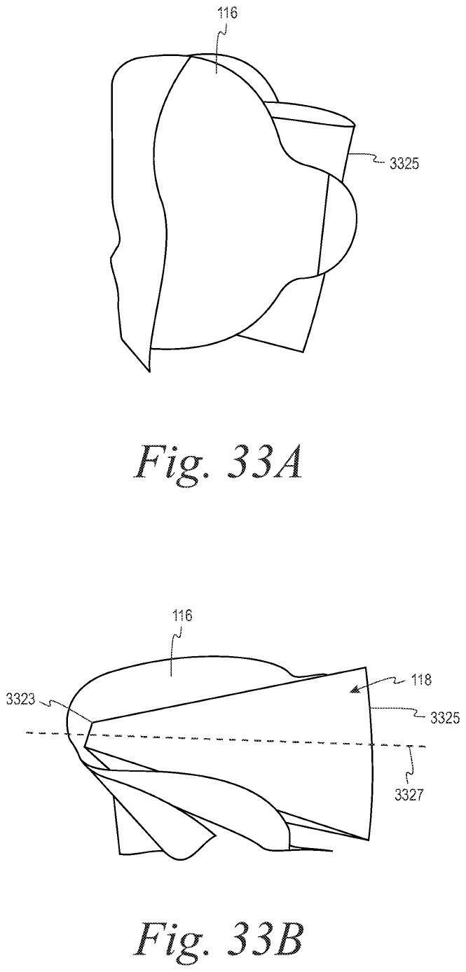

Referring now to FIGS. 33A-43E, additional or alternative implementations for the applicator 118, which can be used with the fecal collection systems 100, 600 are shown and described, according to further examples. As described above, the applicator 118 can assist in coupling the user interface 116 to the skin of the user.

FIGS. 33A-33C depict an implementation in which the applicator 118 is formed from a foam material and has a wedge shape. For example, the applicator 118 can have a proximal side 3323 that engages the distal side of the user interface 116, and a distal side 3323 that is opposite of the proximal side 3323. The applicator 118 can taper inwardly toward a center axis 3327 along a direction from the distal side 3325 toward the proximal side (e.g., the applicator 118 is wider at the distal side 3325 than the proximal side 3323). In this arrangement, the wedge shape of the applicator 118 made from the foam material can conform well to an anatomical shape of the buttocks when using the applicator 118 to couple the user interface 116 to the user.

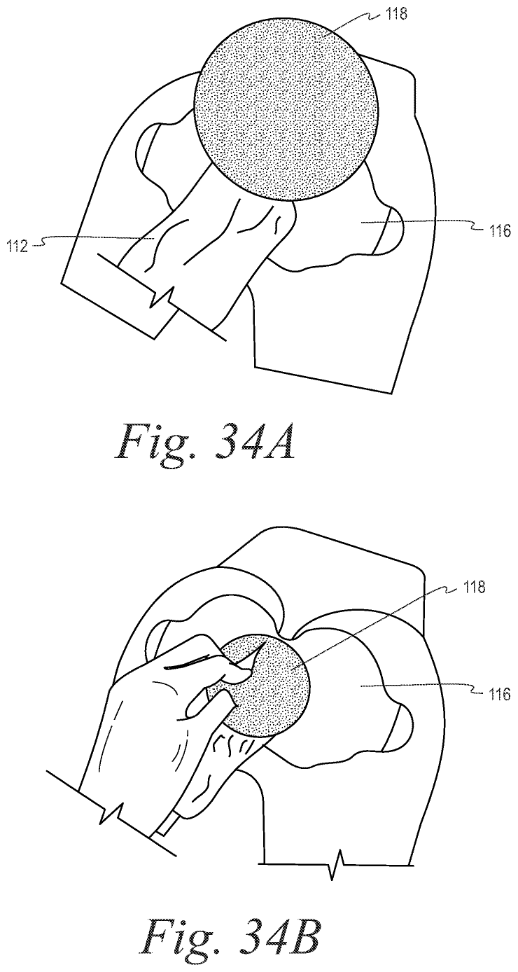

FIGS. 34A-34B depict an implementation in which the applicator 118 is formed from a foam material and has a sphere shape, according to an example. The sphere shape can help to create surface area for the applicator 118 to conform the adhesive of the user interface 116 against the anatomy of the user. Additionally, the applicator 118 formed from the foam material and having the sphere shape can help to concentrate an applied force at the anal opening when using the applicator 118 to couple the user interface 116 to the user. The applicator 118 shown in FIGS. 34A-34B can additionally or alternatively be particularly suitable for patting the adhesive of the user interface 116 at the anus.

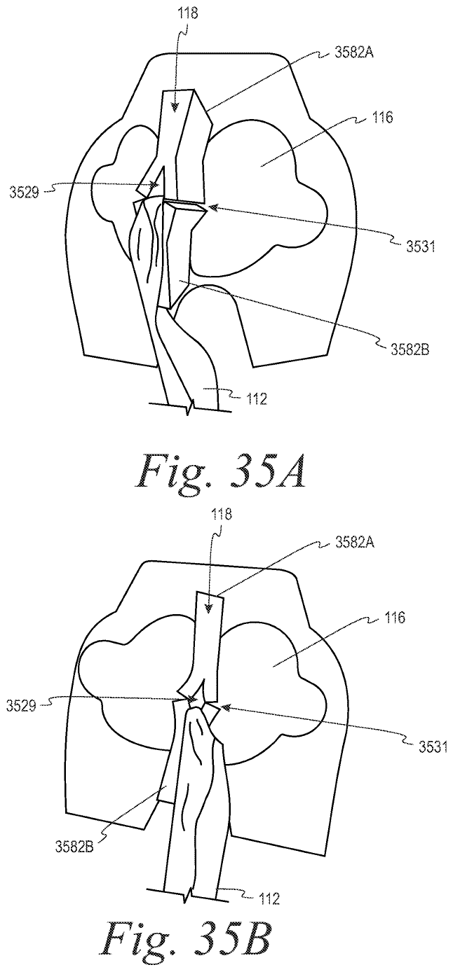

FIGS. 35A-35B depict an implementation in which the applicator 118 is formed from a foam material and has an elongated shape with an aperture 3529 extending between a proximal side and a distal side of the applicator 118. The drainage conduit 112 can extend through the aperture 3529. As shown in FIGS. 35A-35C, the applicator 118 can be elongated in a dimension extending between a superior side 3582A and an inferior side 3582B. This can provide the applicator 118 with a surface area that can facilitate gripping the applicator 118 while allowing the applicator 118 to have a relatively narrow width so as to fit between the buttocks of the user. In some implementations, the applicator 118 can slide along the drainage conduit 112. This can allow the applicator 118, the drainage conduit 112, and the user interface 112 to be packaged as assembly to simplify workflow.

In some implementations, the applicator 118 can additionally or alternatively be configured to be decoupled from the drainage conduit 112 after the user interface 116 has been coupled to the user. For instance, the applicator 118 can include one or more lines of weakness 3331 (e.g., one or more cut lines and/or one or more score lines) that can facilitate tearing the applicator 118 at the aperture 3329 and removing the applicator 118 from the drainage conduit 112.

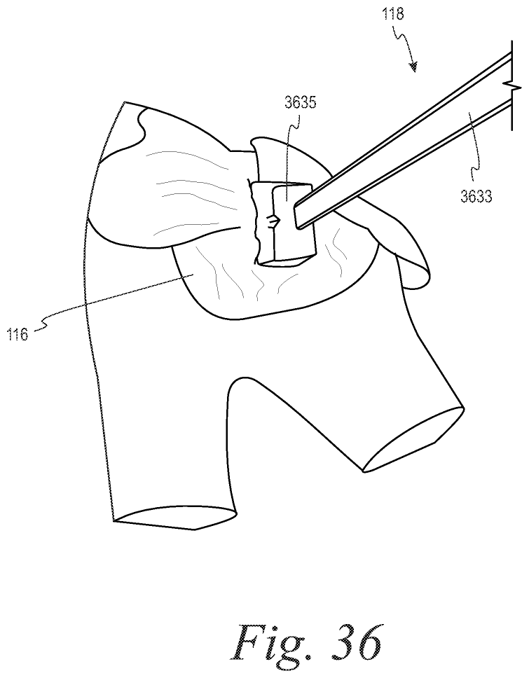

FIG. 36 depicts an implementation in which the applicator 118 includes a shaft 3633 extending between a proximal end and a distal end, and a foam pad 3635 at the proximal end of the shaft 3633. Within examples, the shaft 3633 can be rigid along an axial dimension between the proximal end and the distal end. This can allow the shaft 3633 to apply axial pressure to the user interface 116 via the foam pad 3635. The shaft 3633 can be elongated in the axial dimension. This can allow the clinician to grip the applicator 118 at a location that is farther away from the anus of the user as compared to other implementations of the applicator described herein. Within examples, the foam pad 3635 can be configured to conform to a shape of the anatomy of the user when the foam pad 3635 presses the user interface 116 against the anatomy of the user. This can help to improve adhesive of the user interface 116 to the user.

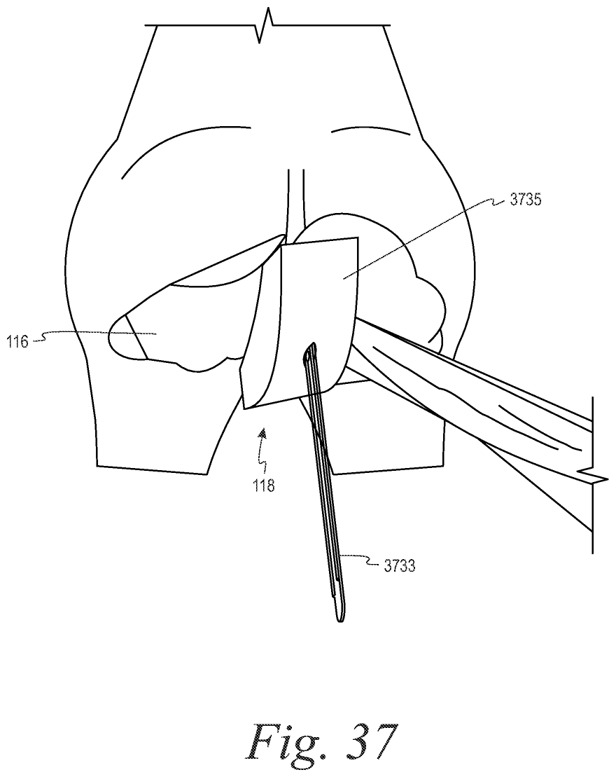

FIG. 37 depicts another implementation in which the applicator 118 includes a shaft 3733 extending between a proximal end and a distal end, and a foam pad 3735 at the proximal end of the shaft 3733. The applicator 118 shown in FIG. 37 is substantially similar or identical to the applicator 118 shown in FIG. 36, except the foam pad 3735 is curved for the applicator 118 shown in FIG. 37. The curved shape of the foam pad 3735 can help to better conform the applicator 118 and the user interface 116 to the anatomy of the user as compared to the implementation shown in FIG. 36.

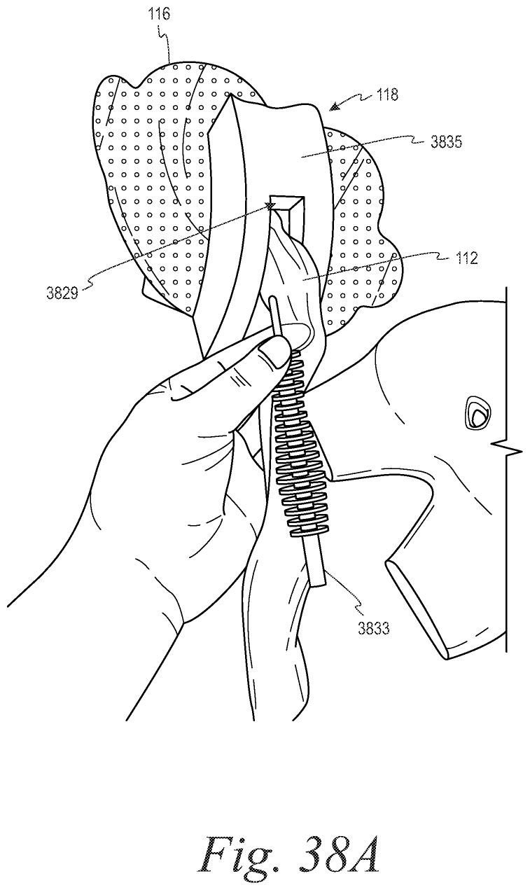

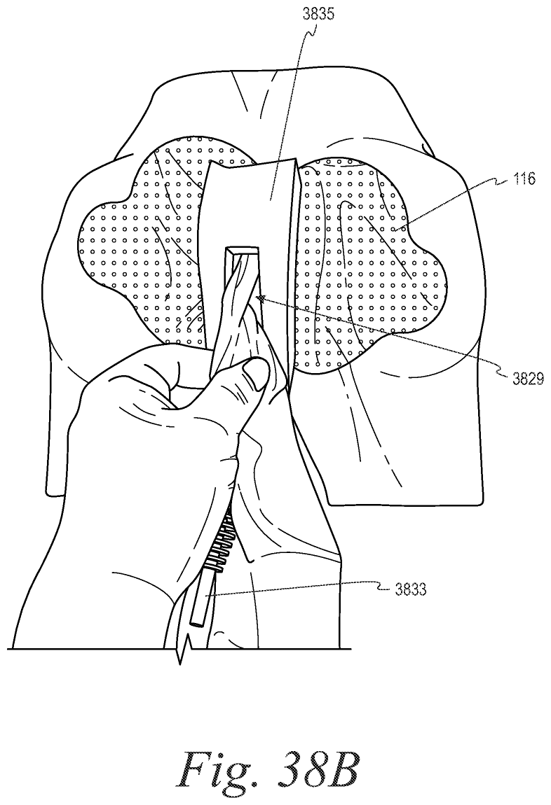

FIG. 38 depicts another implementation in which the applicator 118 includes a shaft 3833 extending between a proximal end and a distal end, and a foam pad 3835 at the proximal end of the shaft 3833. In this implementation, the foam pad 3835 can include an aperture 3829, and the drainage conduit 112 can extend through the aperture 3829. In this example, the aperture 3829 can allow for improved visualization of the user interface 116 through the aperture 3829 of the applicator 118. Also, in this example, the foam pad 3835 has a curved shape, as described above.

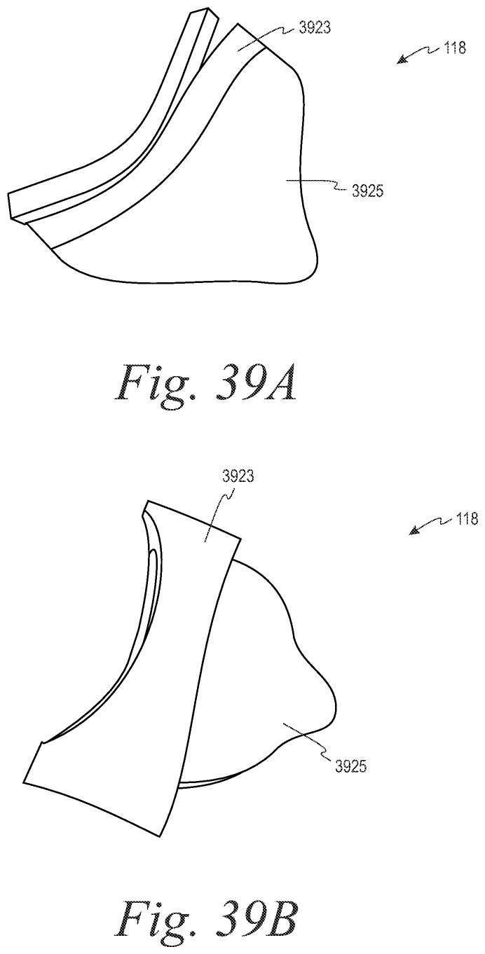

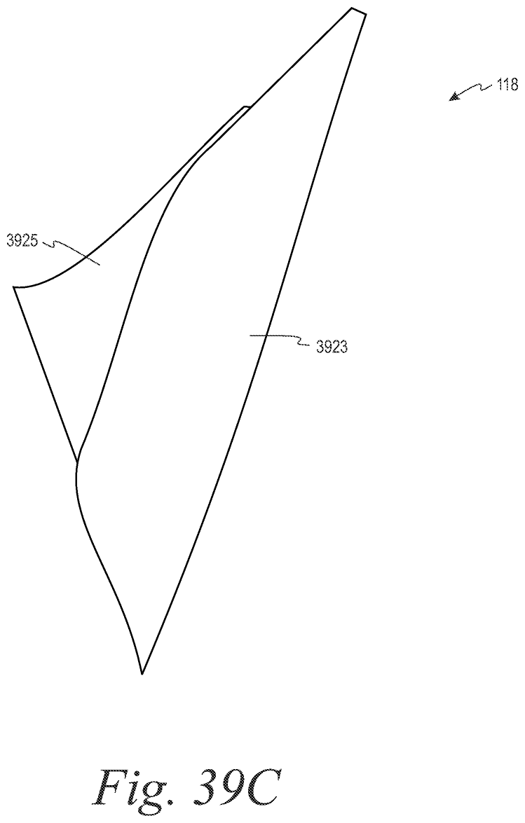

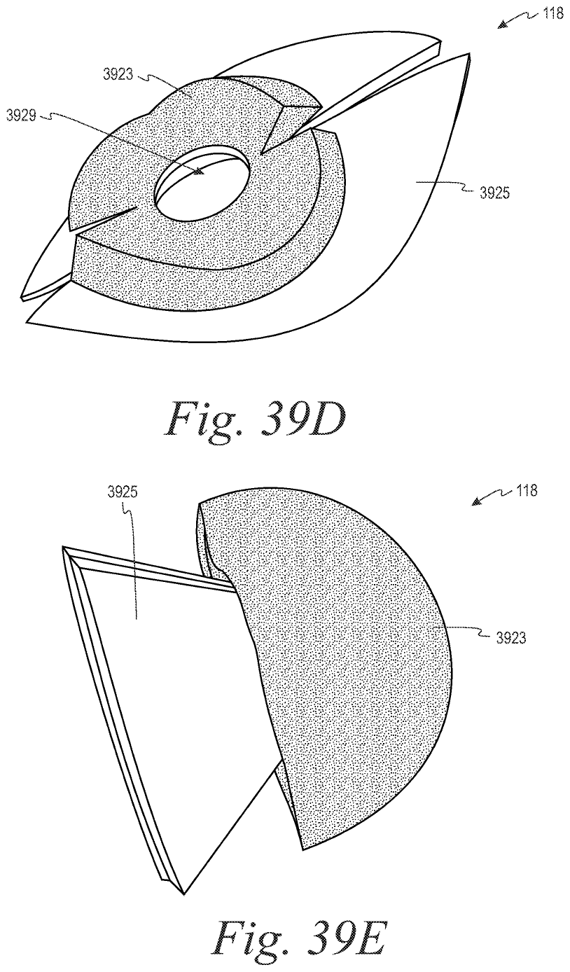

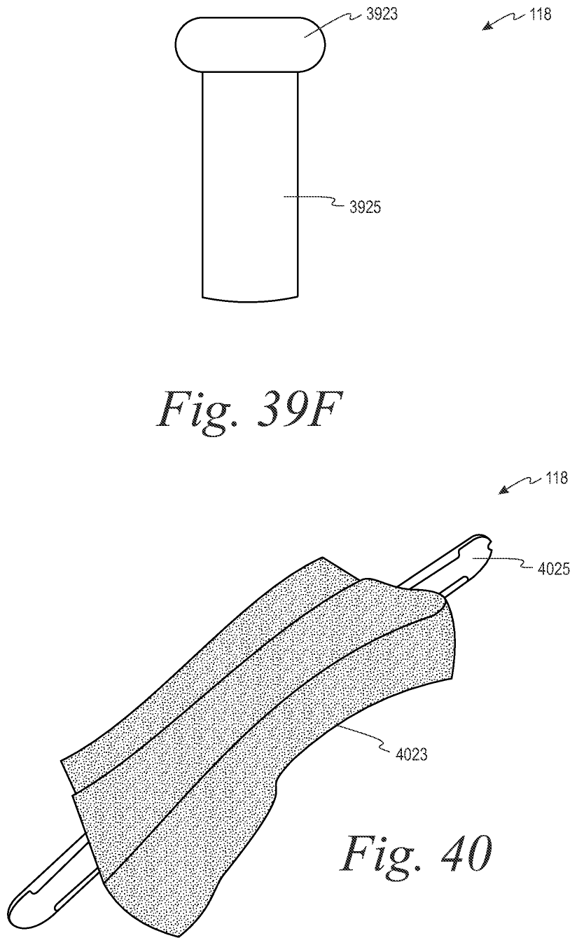

FIGS. 39A-39F depict additional implementations in which the applicator 118 includes a proximal portion 3923 that is a foam material, and a distal portion 3925 that is a rigid material. The foam of the proximal portion 3923 can conform to the anatomy of the user, whereas the distal portion 3925 can provide structural rigidity to press the proximal portion 3923 against the user interface 116 being adhered to the user. As an example, the rigid material of the distal portion 3925 can be a cardstock material. In FIG. 39A, the foam of the proximal portion 3923 covers a proximal-most edge of the applicator 118. In FIG. 39B, the foam of the proximal portion 3923 has a concave shape. This can help to create more pressure above and below the anus to provide a better seal between the user interface 116 and the user. In FIG. 39C, the distal portion 3925 has a triangular shape when folded, and the foam of the proximal portion 3923 extends over a majority of a surface area of the distal portion 3925. This can enhance a structural support for the foam of the proximal portion 392. In FIG. 39D, the distal portion 3925 has a clam shell shape with an aperture 3929, and the foam of the proximal portion 3923 extends around the aperture 3929. In FIG. 39E, the foam of the proximal portion 3923 is semi-spherical, and the distal portion 3925 is formed from a more rigid foam material. In FIG. 39F, the distal portion 3925 is a rigid tube, and the proximal portion 3923 has a ring shape.

FIG. 40 depicts another implementation in which the applicator 118 includes a foam portion 4023 and a malleable shaft 4025 coupled to the foam portion 4023. The foam portion 4023 can be configured to conform to the anatomy of the user when the foam portion 4023 is pressed against the user interface 116 to adhere the user interface 116 to the user. The malleable shaft 4025 is bendable in response to a bending force without breaking. This can allow a clinician to modify the shape of the malleable shaft 4025 and the foam portion 4023 according to a specific anatomy of a particular user.

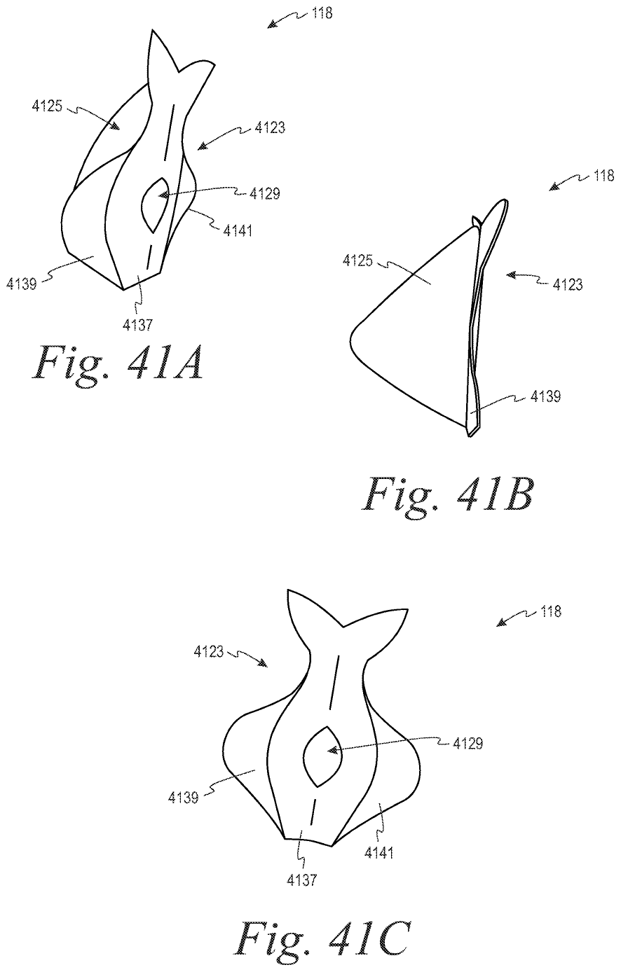

FIGS. 41A-41C depict another implementation in which the applicator 118 includes a proximal portion 4123 and a distal portion 4125. The proximal portion 4123 includes a center portion 4137, a first lateral wing portion 4139 on a first lateral side of the center portion 4138, and a second lateral wing portion 4141 on a second lateral side of the center portion 4138. The distal portion 4125 extends distally from the center portion 4138 of the proximal portion 4123. The proximal portion 4123 can be formed from a soft, pliable foam material, and the distal portion 4125 can be formed of a more rigid material (e.g., a rigid foam and/or a cardstock). The proximal portion 4123 can include an aperture 4129 at the center portion 4137. In this arrangement, the distal portion 4125 can assist in pressing the proximal portion 4123 against the user interface 116 and the user, and the proximal portion 4123 can apply pressure to an area around the anus when the aperture 4129 is aligned with the anus.

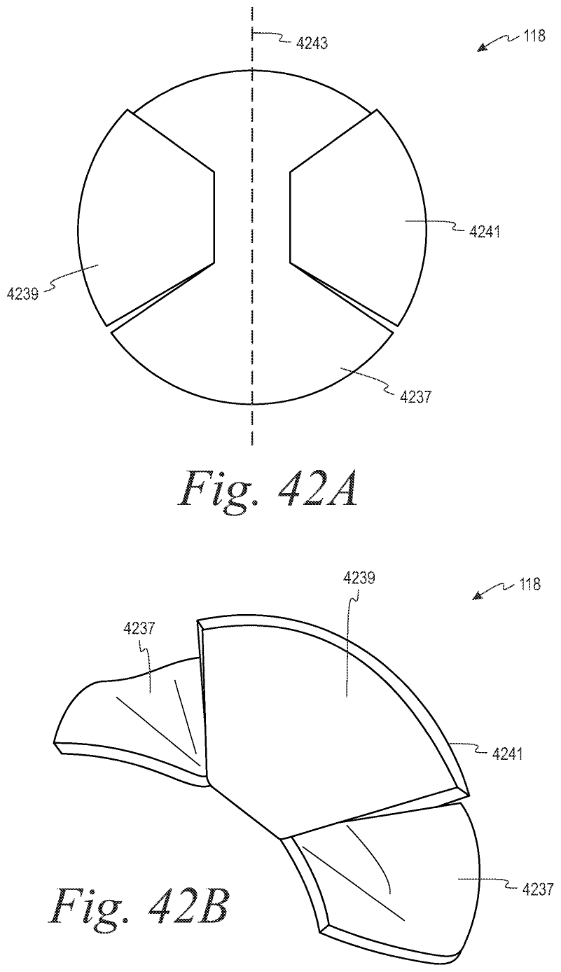

FIGS. 42A-42B depict another implementation in which the applicator 118 includes a center portion 4237, a first flap 4237 on a first lateral side of the center portion 4237, and a second flap 4239 on a second lateral side of the center portion 4237. As shown in FIG. 42A, the center portion 4237, the first flap 4237, and the second flap 4239 can have a circular shape when the applicator 118 is laid flat. As shown in FIG. 42B, the applicator 118 can be folded at a center axis 4243, and the first flap 4239 and the second flap 4241 can be used as a handle for the applicator 118. While holding a distal portion of the first flap 4239 and the second flap 4231, the center portion 4237 can be pressed against the user interface 116 and the user to couple the user interface 116 to the user.

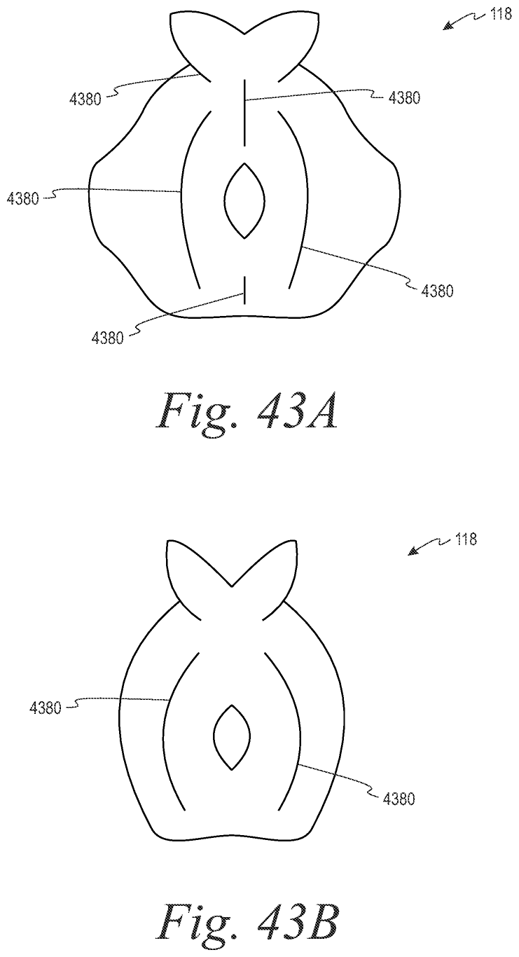

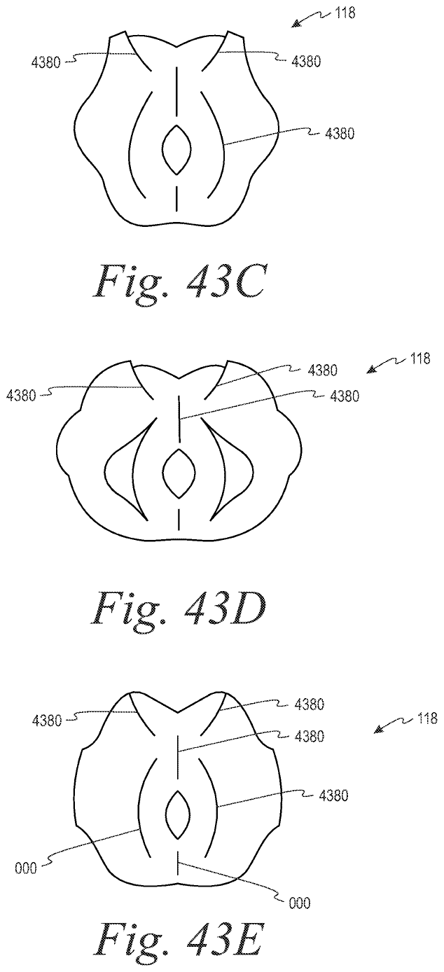

FIGS. 43A-43E depict additional implementations of the applicator 118, according to additional examples. The applicators 118 shown in FIGS. 43A-43E are substantially similar or identical to the applicator 118 shown and described above with respect to FIGS. 25A-26D, except the fold lines 4380 (e.g., cut lines) have a different configuration to show another example of the three-dimensional geometries that can be provided by the central portion for assisting in coupling the user interface 116 to the skin of the user.

The description of the different advantageous arrangements has been presented for purposes of illustration and description, and is not intended to be exhaustive or limited to the embodiments in the form disclosed. Many modifications and variations will be apparent to those of ordinary skill in the art. Further, different advantageous embodiments may describe different advantages as compared to other advantageous embodiments. The embodiment or embodiments selected are chosen and described in order to explain the principles of the embodiments, the practical application, and to enable others of ordinary skill in the art to understand the disclosure for various embodiments with various modifications as are suited to the particular use contemplated.

What is claimed is:

1. A fecal collection system, comprising:
a user interface having a proximal side and a distal side, wherein the proximal side is configured to couple to skin of a user, wherein the user interface comprises an aperture extending through the proximal side and the distal side;
an applicator coupled to the distal side of the user interface, wherein the applicator comprises an aperture extending through a proximal side and a distal side of the applicator, wherein the aperture of the applicator overlaps with the aperture of the user interface, wherein the applicator further comprises:
a central portion extending between a superior side of the applicator and an inferior side of the applicator, wherein the central portion includes the aperture of the applicator,
a first lateral portion on a first lateral side of the central portion, and
a second lateral portion on a second lateral side of the central portion,
a plurality of fold lines about which the first lateral portion and the second lateral portion are configured to fold relative to the central portion,
wherein a first subset of the plurality of fold lines are between the central portion and the first lateral portion,
wherein a second subset of the plurality of fold lines are between the central portion and the second lateral portion,
wherein, along a width extending between a first lateral side of the of the applicator and a second lateral side of the applicator, the first subset of the plurality of fold lines and the second subset of the plurality of fold lines are separated by a non-zero distance from each other, and
wherein the first lateral portion and the second lateral portion are configured to move about the first subset of the plurality of fold lines and the second subset of the plurality of fold lines, respectively, to actuate the applicator between an extended state and a folded state,
a drainage conduit extending between a proximal end and a distal end, wherein the proximal end is configured to couple to the distal side of the user interface, wherein the drainage conduit defines a lumen configured to guide feces from the proximal end to the distal end; and
a collection reservoir coupled to the distal end of the drainage conduit.

2. The fecal collection system of claim 1, wherein the plurality of fold lines are formed by respective partial cuts that extend partially through a thickness of the applicator, and
wherein the thickness of the applicator extends in a dimension between the proximal side and the distal side of the applicator.

3. The fecal collection system of claim 2, wherein the respective partial cuts each extend through approximately 75 percent of the thickness of the applicator.

4. The fecal collection system of claim 1, wherein the applicator is formed from at least one material selected from a group consisting of: a foam, cardstock, cardboard, and chipboard.

5. The fecal collection system of claim 4, wherein the applicator is formed from the foam.

6. The fecal collection system of claim 1, wherein the plurality of fold lines (i) extend from the aperture of the applicator to the superior side of the applicator and (ii) extend from the aperture of the applicator to the inferior side of the applicator.

7. The fecal collection system of claim 6, wherein, when the applicator is in the folded state, a width of the central portion at the aperture of the applicator is greater than 50 percent of the width of the aperture of the applicator when the applicator is in the extended state.

8. The fecal collection system of claim 6, wherein a center axis of the applicator (i) extends in a dimension between the superior side and the inferior side and (ii) through the aperture of the applicator,
wherein the plurality of fold lines comprise a first fold line and a second fold line that (i) extend in a superior direction from opposing sides of the aperture of the applicator towards the superior side, and (ii) taper toward the center axis of the applicator along the superior direction, and
wherein the plurality of fold lines comprise a third fold line and a fourth fold line that (i) extend in an inferior direction from opposing sides of the aperture of the applicator towards the inferior side, and (ii) taper toward the center axis of the applicator along the inferior direction.

9. The fecal collection system of claim 8, wherein the plurality of fold lines further comprise a fifth fold line that extends from the first fold line to the superior side and a sixth fold line that extends from the second fold line to the superior side, and
wherein the fifth fold line and the sixth fold line diverge away from the center axis of the applicator along the superior direction.

10. The fecal collection system of claim 9, wherein the plurality of fold lines further comprise a seventh fold line that extends from the third fold line to the inferior side and a eighth fold line that extends from the fourth fold line to the inferior side, and
wherein the seventh fold line and the eighth fold line diverge away from the center axis of the applicator along the inferior direction.

11. The fecal collection system of claim 1, wherein a first lateral side and a second lateral side of the applicator each comprise a plurality of ridges that are configured to indicate a grip location for holding the applicator when the applicator is in the folded state.

12. The fecal collection system of claim 1, wherein when the applicator is in the folded state, the distal side at the first lateral portion faces the distal side at the second lateral portion.

13. The fecal collection system of claim 1, wherein the drainage conduit extends through the aperture of the applicator.

14. The fecal collection system of claim 1, wherein the user interface comprises an adhesive sheet including an adhesive on the proximal side, and
wherein the adhesive sheet defines the aperture of the user interface and is axially aligned with a lumen of the drainage conduit.

15. The fecal collection system of claim 1, wherein the plurality of fold lines comprise respective partial cuts through a thickness of the applicator, and wherein the thickness of the applicator extends in a dimension between the proximal side and the distal side of the applicator.

16. The fecal collection system of claim 1, wherein the plurality of fold lines comprise respective complete cuts through a thickness of the applicator, and wherein the thickness of the applicator extends in a dimension between the proximal side and the distal side of the applicator.

17. The fecal collection system of claim 1, wherein the plurality of fold lines extend entirely around the aperture of the applicator such that the aperture is entirely positioned within the central portion.

18. The fecal collection system of claim 17, wherein a center axis of the applicator (i) extends in a dimension between the superior side and the inferior side and (ii) through the aperture of the applicator,
wherein the plurality of fold lines comprise a first fold line and a second fold line that extend around the aperture.

19. The fecal collection system of claim 18, wherein the first fold line and the second fold line define an ovular shape around the aperture.

20. The fecal collection system of claim 18, wherein the plurality of fold lines further comprise a third fold line that extends from the first fold line to the superior side and a fourth fold line that extends from the second fold line to the superior side.

21. The fecal collection system of claim 20, wherein the plurality of fold lines further comprise a fifth fold line that extends from the first fold line to the inferior side and a sixth fold line that extends from the second fold line to the inferior side.

22. A method of using a fecal collection system, comprising:
positioning, using an applicator, a user interface at an anus of a user, wherein the user interface has a proximal side and a distal side, wherein the proximal side is configured to couple to skin of a user, wherein the user interface comprises an aperture extending through the proximal side and the distal side, wherein the applicator is coupled to the distal side of the user interface, wherein the applicator comprises an aperture extending through a proximal side and a distal side of the applicator, wherein the applicator further comprises:
a central portion extending between a superior side of the applicator and an inferior side of the applicator, wherein the central portion includes the aperture of the applicator,
a first lateral portion on a first lateral side of the central portion, and
a second lateral portion on a second lateral side of the central portion,
a plurality of fold lines about which the first lateral portion and the second lateral portion are configured to fold relative to the central portion,
wherein a first subset of the plurality of fold lines are between the central portion and the first lateral portion,
wherein a second subset of the plurality of fold lines are between the central portion and the second lateral portion,
wherein, along a width extending between a first lateral side of the of the applicator and a second lateral side of the applicator, the first subset of the plurality of fold lines and the second subset of the plurality of fold lines are separated by a non-zero distance from each other, and
wherein the first lateral portion and the second lateral portion are configured to move about the first subset of the plurality of fold lines and the second subset of the plurality of fold lines, respectively, to actuate the applicator between an extended state and a folded state,
coupling the user interface to skin of the user by using the applicator, while the applicator in the folded state, to press the user interface against the skin of the user; and
after coupling the user interface to the skin of the user, removing the applicator from the user interface.

23. The method of claim 22, wherein the plurality of fold lines are formed by respective partial cuts that extend partially through a thickness of the applicator, and
wherein the thickness of the applicator extends in a dimension between the proximal side and the distal side of the applicator.

24. The method of claim 22, wherein positioning the user interface at the anus of the user comprises gripping the applicator at a plurality of ridges on a first lateral side and a second lateral side of the applicator each when the applicator is in the folded state.

25. The method of claim 22, further comprising actuating the applicator from the extended state to the folded state by folding along the plurality of fold lines the applicator such that the distal side at the first lateral portion faces the distal side at the second lateral portion.

26. The method of claim 22, wherein coupling the user interface to skin of the user comprises coupling an adhesive sheet of the user interface to the skin of the user.

27. The method of claim 22, wherein, when the applicator is in the folded state, a width of the central portion at the aperture of the applicator is greater than 50 percent of the width of the aperture of the applicator when the applicator is in the extended state.

28. A fecal collection system, comprising:
a user interface having a proximal side and a distal side, wherein the proximal side is configured to couple to skin of a user, wherein the user interface comprises an aperture extending through the proximal side and the distal side;
an applicator coupled to the distal side of the user interface, wherein the applicator comprises an aperture extending through a proximal side and a distal side of the applicator, wherein the aperture of the applicator overlaps with the aperture of the user interface;
a drainage conduit extending between a proximal end and a distal end, wherein the proximal end is configured to couple to the distal side of the user interface, wherein the drainage conduit defines a lumen configured to guide feces from the proximal end to the distal end; and
a collection reservoir coupled to the distal end of the drainage conduit,
wherein the collection reservoir comprises a distal end, a proximal end, a first lateral side that extends between the distal end and the proximal end, and a second lateral side that extends between the distal end and the proximal end and is opposite the first lateral side, and
wherein proximal end defines an aperture for receiving feces into the collection reservoir.

29. The fecal collection system of claim 28, wherein the first lateral side comprises a first distal portion, a first proximal portion, and a first corner at an interface between the first distal portion and the first proximal portion,
wherein the second lateral side comprises a second distal portion, a second proximal portion, and a second corner at an interface between the second distal portion and the second proximal portion, and
wherein the first corner and the second corner are configured such that the first proximal portion and the second proximal portion taper inwardly from the first distal portion and the second distal portion, respectively, along a proximal direction towards the proximal end.

30. The fecal collection system of claim 29, wherein the first corner and the second corner provide a visual indicator of a fill line to indicate that the collection reservoir has been filled with a predetermined amount of feces.

31. The fecal collection system of any one of claims 29-30, wherein a width of the collection reservoir can be constant over a height of the first distal portion and the second distal portion.

32. The fecal collection system of any one of claims 29-31, wherein a ratio of (i) the height of the first distal portion and the second distal portion relative to (ii) the height of the first proximal portion and the second proximal portion is approximately 5:4.

33. The fecal collection system of any one of claims 28-32, wherein the collection reservoir further comprises a port and a cap, wherein the port defines an opening in the collection reservoir through which feces can be received from the drainage conduit, and wherein the cap is configured to removably couple to the port at the opening.

34. The fecal collection system of claim 33, wherein the cap is configured to couple to the port by a bayonet-type coupling.

35. The fecal collection system of any one of claims 33-34, wherein the cap is coupled to the port by a connector portion in a form of a leash.

36. The fecal collection system of any one of claims 33-35, wherein the port comprises a hook.

37. The fecal collection system of any one of claims 29-36, wherein the collection reservoir further comprises a second one-way valve that is configured to allow feces to flow from the drainage conduit to the collection reservoir and inhibit the feces from flowing from the collection reservoir to the drainage conduit, and
wherein the second one-way valve extends from the proximal end proximally into an internal cavity of the collection reservoir, wherein.

38. The fecal collection system of claim 37, wherein the second one-way valve includes a distal end, a proximal end, a first lateral side that extends between the distal end and the proximal end, and a second lateral side that extends between the distal end and the proximal end and is opposite the first lateral side,
wherein the distal end and the proximal end define apertures for receiving the feces through the second one-way valve and into the collection reservoir, respectively.

39. The fecal collection system of any one of claims 37-38, wherein the proximal end of the second one-way valve is coupled to the proximal end of the collection reservoir 114 such that an opening at the proximal end of the second one-way valve is always open.

40. The fecal collection system of any one of claims 37-39, wherein the distal end of the second one-way valve is disposed freely in the internal cavity of the collection reservoir such that the distal end collapses on itself when no feces is passing through a passage of the second one-way valve in a distal direction from the distal end towards the proximal end.

41. The fecal collection system of any one of claims 37-40, wherein the first lateral side comprises a first distal portion, a first proximal portion, and a first corner at an interface between the first distal portion and the first proximal portion,
wherein the second lateral side comprises a second distal portion, a second proximal portion, and a second corner at an interface between the second distal portion and the second proximal portion, and
wherein he first corner and the second corner are configured such that the first distal portion and the second distal portion taper inwardly from the first proximal portion and the second proximal portion, respectively, along a proximal direction towards the distal end.

42. The fecal collection system of claim 41, wherein the first lateral side further comprises a first non-tapered section that is between the first proximal portion and the proximal end, and the second lateral side comprises a second non-tapered section that is between the second proximal portion and the proximal end.

43. The fecal collection system of any one of claims 38-42, wherein the distal end of the second one-way valve can be positioned proximally of the first corner and the second corner of the collection reservoir.

44. The fecal collection system of any one of claims 28-43, wherein the user interface comprises:
one or more adhesive sheets on a proximal side of the user interface;
a first lateral release liner removably coupled to a first lateral portion of the one or more adhesive sheets;
a second lateral release liner removably coupled to a second lateral portion of the one or more adhesive sheets; and
a middle release liner removably coupled to a middle portion of the one or more adhesive sheets,
wherein the middle release liner is removably coupled to the one or more adhesive sheets between the first lateral release liner and the second lateral release liner.
